SEQUENCE LISTING (43 sequences)

SEQ ID NO: 1 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 2 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 3 - DNA, 15 bp, synthetic construct
  source 1..15: mol_type = other DNA
ttcaaggacgacggc

SEQ ID NO: 4 - protein, 5 aa, synthetic construct
FKDDG

SEQ ID NO: 5 - DNA, 15 bp, synthetic construct
  source 1..15: mol_type = other DNA
gccgaggtgaagttc

SEQ ID NO: 6 - protein, 5 aa, synthetic construct
AEVKF

SEQ ID NO: 7 - DNA, 15 bp, synthetic construct
  source 1..15: mol_type = other DNA
ttcaaggaggacggc

SEQ ID NO: 8 - protein, 5 aa, synthetic construct
FKEDG

SEQ ID NO: 9 - DNA, 15 bp, synthetic construct
  source 1..15: mol_type = other DNA
aacatcgaggacggc

SEQ ID NO: 10 - protein, 5 aa, synthetic construct
NIEDG

SEQ ID NO: 11 - DNA, 15 bp, synthetic construct
  source 1..15: mol_type = other DNA
gacgaggtcgacggc

SEQ ID NO: 12 - DNA, 15 bp, synthetic construct
  source 1..15: mol_type = other DNA
gacgaggtggacggc

SEQ ID NO: 13 - protein, 5 aa, synthetic construct
DEVDG

SEQ ID NO: 14 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacgacgaggtggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 15 - DNA, 35 bp, synthetic construct
  source 1..35: mol_type = other DNA
aataccactgagatctatggtgagcaagggcgagg

SEQ ID NO: 16 - DNA, 40 bp, synthetic construct
  source 1..40: mol_type = other DNA
agagggaaaaagatctttacttgtacagctcgtccatgcc

SEQ ID NO: 17 - DNA, 35 bp, synthetic construct
  source 1..35: mol_type = other DNA
gggcctcgagagatctatggtgagcaagggcgagg

SEQ ID NO: 18 - DNA, 37 bp, synthetic construct
  source 1..37: mol_type = other DNA
ctccgctgccagatctcttgtacagctcgtccatgcc

SEQ ID NO: 19 - DNA, 20 bp, synthetic construct
  source 1..20: mol_type = other DNA
cccctatgtccacttcagga

SEQ ID NO: 20 - DNA, 20 bp, synthetic construct
  source 1..20: mol_type = other DNA
aggatcctctctggctccat

SEQ ID NO: 21 - DNA, 20 bp, synthetic construct
  source 1..20: mol_type = other DNA
gttcaccttgatgccgttct

SEQ ID NO: 22 - DNA, 20 bp, synthetic construct
  source 1..20: mol_type = other DNA
agaagaacggcatcaaggtg

SEQ ID NO: 23 - DNA, 20 bp, synthetic construct
  source 1..20: mol_type = other DNA
cttgtacagctcgtccatgc

SEQ ID NO: 24 - DNA, 20 bp, synthetic construct
  source 1..20: mol_type = other DNA
gagccacatcgctcagacac

SEQ ID NO: 25 - DNA, 20 bp, synthetic construct
  source 1..20: mol_type = other DNA
aaggggtcattgatggcaac

SEQ ID NO: 26 - DNA, 4833 bp, synthetic construct
  source 1..4833: mol_type = other DNA
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgcggccgcgctagccagctggatatcgagctctgatcaggtaccaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 27 - DNA, 5559 bp, synthetic construct
  source 1..5559: mol_type = other DNA
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgcggccgcgctagccagctggatatcgagctctgatcaggtaccaattcactcctcaggtgcaggctgcctatcagaaggtggtggctggtgtggccaatgccctggctcacaaataccactgagatctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcgacgaggtcgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 28 - DNA, 5559 bp, synthetic construct
  source 1..5559: mol_type = other DNA
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgcggccgcgctagccagctggatatcgagctctgatcaggtaccaattcactcctcaggtgcaggctgcctatcagaaggtggtggctggtgtggccaatgccctggctcacaaataccactgagatctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgacgaggtggacggcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 29 - DNA, 5559 bp, synthetic construct
  source 1..5559: mol_type = other DNA
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgcggccgcgctagccagctggatatcgagctctgatcaggtaccaattcactcctcaggtgcaggctgcctatcagaaggtggtggctggtgtggccaatgccctggctcacaaataccactgagatctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacgacgaggtggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 30 - DNA, 5559 bp, synthetic construct
  source 1..5559: mol_type = other DNA
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgcggccgcgctagccagctggatatcgagctctgatcaggtaccaattcactcctcaggtgcaggctgcctatcagaaggtggtggctggtgtggccaatgccctggctcacaaataccactgagatctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacgacgaggtggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 31 - DNA, 6602 bp, synthetic construct
  source 1..6602: mol_type = other DNA
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcagaattaaccctcactaaagggactagtcctgcaggtttaaacgaattcgccctttgctttctctgaccagcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggcaagcttctgacctcttctcttcctcccacagggcctcgagagatctggcagcggagagggcagaggaagtcttctaacatgcggtgacgtggaggagaatcccggccctaggctcgagatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtcgacagtactaagctttactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctctaagggcaagggcgaattcgcggccgctaaattcaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 32 - DNA, 7325 bp, synthetic construct
  source 1..7325: mol_type = other DNA
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcagaattaaccctcactaaagggactagtcctgcaggtttaaacgaattcgccctttgctttctctgaccagcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggcaagcttctgacctcttctcttcctcccacagggcctcgagagatctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacgacgaggtggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagagatctggcagcggagagggcagaggaagtcttctaacatgcggtgacgtggaggagaatcccggccctaggctcgagatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtcgacagtactaagctttactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctctaagggcaagggcgaattcgcggccgctaaattcaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 33 - protein, 239 aa, synthetic construct
MVSKGEELFTGVVPILVELDGDVNGHKFSVSGEGEGDATYGKLTLKFICTTGKLPVPWPTLVTTLTHGVQCFSRYPDHMKQHDFFKSAMPEGYVQERTIFFKDDGNYKTRAEVKFEGDTLVNRIELKGIDFKEDGNILGHKLEYNFNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 34 - protein, 240 aa, synthetic construct
MLVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK

SEQ ID NO: 35 - protein, 239 aa, synthetic construct
MVSKGEELFTGVVPILVELDGDVNGHKFSVSGEGEGDATYGKLTLKFICTTGKLPVPWPTLVTTLTWGVQCFSRYPDHMKQHDFFKSAMPEGYVQERTIFFKGDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSALSKDPNEKRDHMVLLEFVTAAGITLGMDELYK

SEQ ID NO: 36 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacccacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacttcaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 37 - DNA, 723 bp, synthetic construct
  source 1..723: mol_type = other DNA
atgctagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa

SEQ ID NO: 38 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaagggcgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 39 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacccacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacttcaacagccacaacgtctatatcatggccgacaagcagaagaacggcatcaaggtgaacttcaagatccgccacgacgaggtggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 40 - DNA, 723 bp, synthetic construct
  source 1..723: mol_type = other DNA
atgctagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacgacgaggtggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa

SEQ ID NO: 41 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaagggcgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacgacgaggtggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 42 - DNA, 5559 bp, synthetic construct
  source 1..5559: mol_type = other DNA
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgcggccgcgctagccagctggatatcgagctctgatcaggtaccaattcactcctcaggtgcaggctgcctatcagaaggtggtggctggtgtggccaatgccctggctcacaaataccactgagatctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacccacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacttcaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 43 - DNA, 5559 bp, synthetic construct
  source 1..5559: mol_type = other DNA
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgcggccgcgctagccagctggatatcgagctctgatcaggtaccaattcactcctcaggtgcaggctgcctatcagaaggtggtggctggtgtggccaatgccctggctcacaaataccactgagatctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacccacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacttcaacagccacaacgtctatatcatggccgacaagcagaagaacggcatcaaggtgaacttcaagatccgccacgacgaggtggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

(19) United States
(12) Patent Application Publication
(10) Pub. No.: US 2026/0309716 A1
(43) Pub. Date: Oct. 8, 2026

(54) FLUORESCENT PROTEIN VARIANT FOR OBSERVING CELL DAMAGE, AND DRUG TOXICITY EVALUATION METHOD USING SAME

(71) Applicants: KOREA RESEARCH INSTITUTE OF BIOSCIENCE AND BIOTECHNOLOGY, Daejeon (KR); UNIVERSITY OF ULSAN FOUNDATION FOR INDUSTRY COOPERATION, Ulsan (KR)

(72) Inventors: Sun-Uk KIM, Daejeon (KR); Young-Ho PARK, Daejeon (KR); In-Sung SONG, Seoul (KR); Hae Jun YANG, Daejeon (KR); Dong Gil LEE, Daejeon (KR); Unbin CHAE, Daejeon (KR)

(73) Assignees: KOREA RESEARCH INSTITUTE OF BIOSCIENCE AND BIOTECHNOLOGY, Daejeon (KR); UNIVERSITY OF ULSAN FOUNDATION FOR INDUSTRY COOPERATION, Ulsan (KR)

(21) Appl. No.: 19/481,741

(22) PCT Filed: May 10, 2023
(86) PCT No.: PCT/KR2023/006381

§ 371 Date: Nov. 5, 2025

(51) Int. Cl. G01N 33/58; C07K 14/435; C12N 15/86

(52) U.S. Cl. CPC G01N 33/582; C07K 14/435; C12N 15/86; C12N 2750/14143

(57) ABSTRACT

The present invention relates to a fluorescent protein variant for detection of cell damage, a gene encoding the same, a vector system including the same, a transformed cell using the same, and a use thereof. The transformed cells according to the present invention can monitor cell damage in real time because changes in fluorescence expression levels, which depend on the activity of caspase 3 or caspase 7, can be observed in real time. Furthermore, the transformed cells according to the present invention can be used to evaluate the toxicity of various drugs. Therefore, the present invention can be effectively applied to cell damage research or drug development.

### TECHNICAL FIELD

The present invention relates to a fluorescent protein variant for detection of cell damage, a gene encoding the same, a vector system including the same, a transformed cell using the same, and a use thereof.

### BACKGROUND ART

Known mechanisms of cell death include necrosis and apoptosis. Unlike necrosis, which can be considered a type of “accidental death” caused by external stimuli, apoptosis, a genetically programmed, selective cell death, is known to occur during development and differentiation to eliminate unnecessary components or when cells are severely damaged beyond repair.

Such apoptosis plays a very important role in maintaining the development and homeostasis of living organisms, and can be divided into an extrinsic pathway and an intrinsic pathway. In addition, the two pathways are connected, and substances reacting in one pathway may affect the other pathway (S Elmore et al., Toxicologic Pathology, 35:495-516, 2007).

Apoptosis is induced by the cleavage of various substrates through the activation of caspase enzymes by a protein chain reaction. In particular, among the caspase enzymes, Caspase 3 and Caspase 7 are cysteine-aspartate proteolytic enzymes that are known to recognize and cleave a specific peptide sequence (Asp-Glu-Val-Asp-Gly; DEVDG) (Thornberry, N. A. et al., J. Biol. Chem, 272:17907, 1997).

Using these characteristics of caspase-3 and caspase-7, various methods have been developed to confirm the degree of caspase activation and the progression of apoptosis by linking DEVDG sequences to various probes. However, existing methods have limitations in that they can only confirm the presence or absence of caspase activation, but cannot confirm the progression of apoptosis in real time, and are difficult to implement in cell lines and animal models due to challenges in achieving stable expression in vitro and in vivo. Accordingly, there is a need for a method that enables stable expression both in vitro and in vivo and allows real-time monitoring of apoptosis progression.

### DISCLOSURE OF INVENTION

### Technical Problem

Therefore, the present inventors mutated a portion of the amino acid sequence in the hinge region of a fluorescent protein, which does not affect its function, to DEVDG to prepare a fluorescent protein variant, and to encode the variant of the fluorescent protein, introduced an AAVS1 vector including the gene encoding a variant of the fluorescent protein into a cell line and confirmed that fluorescence expression was changed by the activity of caspase 3 or caspase 7, thereby completing the present invention.

### Solution to Problem

In order to achieve the above-described object, an aspect of the present invention provides a fluorescent protein (FP) variant in which fluorescence expression changes in damaged or dead cells, wherein the fluorescent protein variant comprises, in the hinge region that does not affect the function of the fluorescent protein, an amino acid sequence that is cleaved by a caspase.

Another aspect of the present invention provides a polynucleotide encoding the fluorescent protein variant.

Still another aspect of the present invention provides a recombinant vector including the polynucleotide.

Still another aspect of the present invention provides a composition for detecting cell damage or apoptosis, which includes the fluorescent protein variant, polynucleotide, or recombinant vector as an active ingredient.

Still another aspect of the present invention provides a transformed cell which is transformed with the recombinant vector.

Still another aspect of the present invention provides a method for evaluating drug toxicity, which includes i) treating the transformed cell with a sample; ii) measuring fluorescence expression of the cell treated with the sample; and iii) determining that the sample has cytotoxicity against the cell when the fluorescence expression changes.

### Advantageous Effects of Invention

The fluorescence expression level of the transformed cells according to the present invention that changes according to the activity of caspase 3 or caspase 7 can be confirmed in real time; therefore, through which, it is possible to monitor cell damage in real time. Furthermore, the transformed cells according to the present invention can be used to evaluate the toxicity of various drugs. Accordingly, the present invention can be effectively used for the study of cell damage research or drug development.

### BRIEF DESCRIPTION OF DRAWINGS

FIG. 1 shows the hydrophilic portion of the sequence of a green fluorescent protein analyzed using the Kyte-Doolittle scale (A) and the Hopp-Woods scale (B).

FIG. 2 shows the portion of #1 site mutation of the green fluorescent protein indicated by the sequence in the box and the black area.

FIG. 3 shows the portion of #2 site mutation of the green fluorescent protein indicated by the sequence in the box and the black area.

FIG. 4 shows the portion of #3 site mutation of the green fluorescent protein indicated by the sequence in the box and the black area.

FIG. 5 shows the portion of #4 site mutation of the green fluorescent protein indicated by the sequence in the box and the black area.

FIG. 6 shows the portions of #1 to #4 site mutations of the green fluorescent protein indicated on the nucleotide sequence.

FIG. 7 shows a schematic diagram of the process of preparing pCAGGS vectors into which nucleotide sequences encoding green fluorescent protein variants #1 to #4 are inserted.

FIG. 8 shows a schematic diagram of the pCAGGS vector (4833 bp).

FIG. 9 shows a schematic view of the pCAGGS vector into which a nucleotide sequence encoding the green fluorescent protein variant #1 has been inserted.

FIG. 10 shows a schematic view of the pCAGGS vector into which a nucleotide sequence encoding the green fluorescent protein variant #2 has been inserted.

FIG. 11 shows a schematic view of the pCAGGS vector into which a nucleotide sequence encoding the green fluorescent protein variant #3 has been inserted.

FIG. 12 shows a schematic view of the pCAGGS vector into which a nucleotide sequence encoding the green fluorescent protein variant #4 has been inserted.

FIG. 13 shows images confirming the expression of green fluorescent protein after introducing a pCAGGS vector, into which a nucleotide sequence encoding one of green fluorescent protein variants #1 to #4 was inserted, into HEK293 cells.

FIG. 14 shows a schematic diagram of the process of preparing the AAVS1 SA-EGFP #4 site mutation-2A-puro-pA donor vector.

FIG. 15 shows a schematic diagram of the AAVS1 SA-2A-puro-pA donor vector.

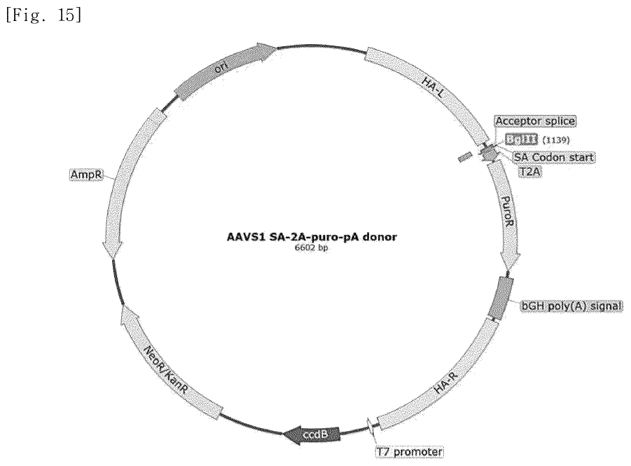

FIG. 16 shows a schematic diagram of the AAVS1 SA-EGFP #4 site mutation-2A-puro-pA donor vector.

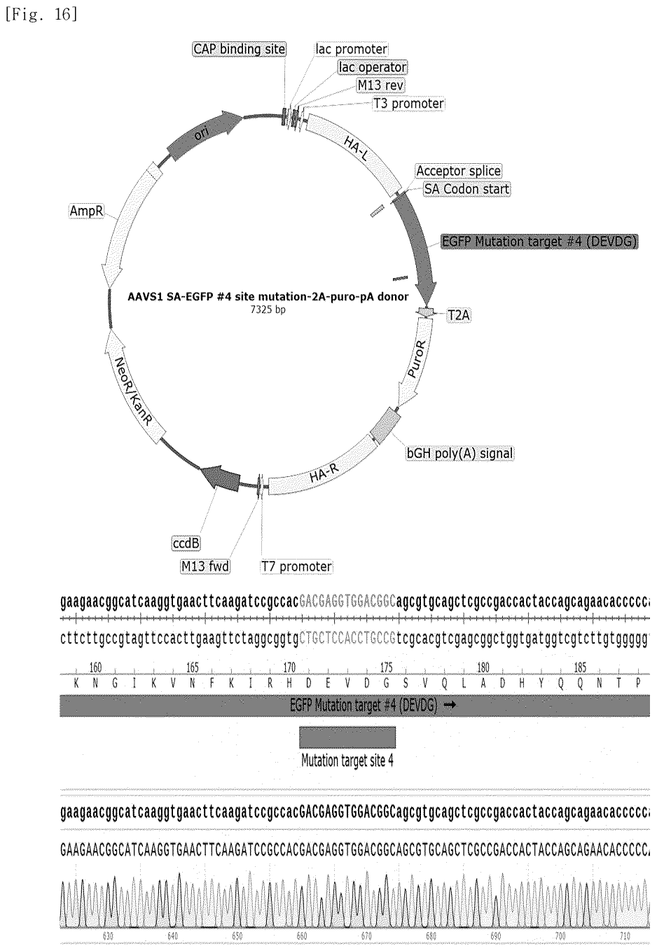

FIG. 17 shows the process of constructing the AAVS1-EGFP #4 (DEVDG) 293 cell line by introducing the AAVS1 SA-EGFP #4 site mutation-2A-puro-pA donor vector into HEK 293 cells.

FIG. 18 shows a schematic diagram of the introduction of AAVS1 SA-EGFP #4 site mutation-2A-puro-pA donor vector and primer positions for genotyping PCR.

FIG. 19 shows the results of analysis by performing genotyping PCR on AAVS1-EGFP #4 (DEVDG) 293 cell line with regard to a case where the vector was not inserted (knock-in; KI) (WT); a case of a heterozygous (hetero) genotype where the vector was inserted into one allele (#3); and a case of a homozygous (homo) genotype where the vector was inserted into both alleles (#8).

FIG. 20 shows the images of AAVS1-EGFP #4-3 (He: hetero genotype) cells and AAVS1-EGFP #4-8 (Ho: homo genotype) cells observed under a fluorescence microscope after treating these cells with Staurosporine (STA) for 48 hours.

FIG. 21 shows the results of flow cytometry analysis after treating AAVS1-EGFP #4-3 (He: hetero genotype) cells and AAVS1-EGFP #4-8 (Ho: homo genotype) cells with staurosporine at a concentration of 1 μM for 48 hours.

FIG. 22 shows the results of Semi RT-PCR analysis after treating AAVS1-EGFP #4-3 (He: hetero genotype) cells and AAVS1-EGFP #4-8 (Ho: homo genotype) cells with staurosporine at a concentration of 1 μM for 48 hours.

FIG. 23 shows the images of AAVS1-EGFP #4-8 (Ho: homo genotype) cells observed under a fluorescence microscope after treating the cells with staurosporine at concentrations of 0.1 μM, 0.25 μM, 0.5 μM, and 1 μM for 48 hours.

FIG. 24 shows the results of flow cytometry analysis after treating AAVS1-EGFP #4-8 (Ho: homo genotype) cells with staurosporine at concentrations of 0.1 μM, 0.25 μM, 0.5 μM, and 1 μM for 48 hours.

FIG. 25 shows the results of Western blot analysis after treating AAVS1-EGFP #4-8 (Ho: homo genotype) cells with staurosporine at concentrations of 0.1 μM, 0.25 μM, 0.5 μM, and 1 μM for 48 hours.

FIG. 26 shows the images of AAVS1-EGFP #4-8 (Ho: homo genotype) cells observed using TUNEL staining and DAPI staining after treating the cells with staurosporine at concentrations of 0.5 μM and 1 μM for 48 hours.

FIG. 27 shows target sequence #4 in the nucleotide sequences of blue fluorescent protein (EBFP), cyan fluorescent protein (ECFP), green fluorescent protein (EGFP), and yellow fluorescent protein (EYFP).

FIG. 28 shows the portion of #4 site mutation of the blue fluorescent protein marked in red.

FIG. 29 shows the portion of #4 site mutation of the yellow fluorescent protein marked in red.

FIG. 30 shows the portion of #4 site mutation of the cyan fluorescent protein marked in red.

FIG. 31 shows a schematic diagram of a pCAGGS vector into which a nucleotide sequence encoding wild-type blue fluorescent protein is inserted.

FIG. 32 shows a schematic diagram of a pCAGGS vector into which a nucleotide sequence encoding a blue fluorescent protein variant #4 is inserted.

FIG. 33 shows images confirming the expression of the blue fluorescent protein after introducing the pCAGGS vector, into which a nucleotide sequence encoding the blue fluorescent protein #4 mutant was inserted, was introduced into HEK293 cells.

FIG. 34 shows images confirming under a fluorescence microscope the expression of the green fluorescent protein after introducing the pCAGGS-EGFP #4 vector into HEK293 cells.

FIG. 35 shows confirming by flow cytometry the expression of the green fluorescent protein after introducing the pCAGGS-EGFP #4 vector into HEK293 cells.

FIG. 36 shows the images observed under a fluorescence microscope after introducing the pCAGFP vector into HEK293 cells and treating the HEK293 cells with staurosporine at a concentration of 1 μM for 48 hours.

FIG. 37 shows the results analyzed by flow cytometry after introducing the pCAGFP vector into HEK293 cells and treating the HEK293 cells with staurosporine at a concentration of 1 M for 48 hours.

FIG. 38 shows real-time cell damage monitoring using real-time imaging fluorescence microscopy for 48 hours at 30-minute intervals in 293 AAVS1-EGFP #4-8 cell lines (A) apoptosis-induced group (staurosporine treatment; +STA, 1 M) and (B) control group (staurosporine untreated; −STA).

FIG. 39 shows fluorescent images and genotyping analysis of a cell line constructed by introducing the AAVS1-EGFP #4 vector into the colorectal cancer cell line SW480, wherein in the genotyping analysis, #3, #4, #6, #9, #13, #15, and #17 have a hetero genotype, and #10 has a wild-type.

FIG. 40 shows fluorescent images and genotyping analysis of a cell line constructed by introducing the AAVS1-EGFP #4 vector into the colorectal cancer cell line SW620, wherein in the genotyping analysis, #2, #3, #6, #7, #8, #9, #10, #13, #14, #24, #27, #28, #35, #36, #37, and #40 have a hetero genotype.

FIG. 41 shows fluorescent images and genotyping analysis of a cell line constructed by introducing the AAVS1-EGFP #4 vector into the colorectal cancer cell line HCT 116, wherein in the genotyping analysis, #1, #3, #4, #5, #6, #10, #13, #14, #16, #17, #19, #20, #21, #22, #23, #24, #26, #29, #31, #34, #37, and #38 have a homo genotype; #2, #7, #8, #27, #28, #30, #32, #33, #35, and #36 have a hetero genotype; and #11 and #15 have a wild-type.

FIG. 42 shows fluorescence images taken 24 and 48 hours after treating the SW480 AAVS1-EGFP #4 cell line with staurosporine at a concentration of 1 μM, and the graph at the bottom shows the relative fluorescence intensity.

FIG. 43 shows fluorescence images taken 24 and 48 hours after treating the SW620 AAVS1-EGFP #4 cell line with staurosporine at a concentration of 1 μM, and the graph at the bottom shows the relative fluorescence intensity.

### BEST MODE FOR CARRYING OUT THE INVENTION

Hereinafter, the present invention will be described in detail.

An aspect of the present invention provides a fluorescent protein (FP) variant in which fluorescence expression changes in damaged or dead cells, wherein the fluorescent protein variant comprises, in the hinge region that does not affect the function of the fluorescent protein, an amino acid sequence that is cleaved by a caspase.

The term “fluorescent protein” of the present invention refers to a protein that emits fluorescence of a specific color in vivo and can observe the action of proteins in vivo. In particular, a fluorescent protein can be tagged to a gene or probe and then injected into a cell, and through this, the intracellular gene expression process, protein synthesis process, cell growth process, etc. can be confirmed.

The fluorescent protein may be green fluorescent protein (GFP), blue fluorescent protein (BFP), yellow fluorescent protein (YFP), or cyan fluorescent protein (CFP), enhanced green fluorescent protein (EGFP), enhanced blue fluorescent protein (EBFP), and may be enhanced yellow fluorescent protein (EYFP) or enhanced cyan fluorescent protein (ECFP), but is not limited thereto, and any protein exhibiting a similar function may be included in that category.

The term “variant” of the present invention includes fragments, and also polypeptides with altered amino acid sequences due to amino acid substitutions, deletions, or insertions. Variants may occur naturally or prepared artificially, and artificial variants may be prepared using mutagenesis techniques known in the art. In addition, variant polypeptides may include conservative or non-conservative amino acid substitutions, deletions, or additions.

The fluorescent protein variant may include an amino acid sequence cleaved by a caspase. Specifically, the amino acid sequence cleaved by a caspase may be aspartic acid-glutamic acid-valine-aspartic acid-glycine (DEVDG) or aspartic acid-glutamic acid-valine-aspartic acid (DEVD), and may be cleaved by caspase 3 or 7. In an embodiment of the present invention, a fluorescent protein variant cleaved by caspase 3 or caspase 7 was prepared in the amino acid sequence of the wild-type fluorescent protein by substituting a partial sequence of the amino acid sequence of the hinge region that does not affect the function of the fluorescent protein, with DEVDG, which is an amino acid sequence cleaved by caspase 3 or caspase 7. Among the amino acid sequences of the wild-type fluorescent protein, amino acid sequences, in which additional substitutions, deletions, or additions were performed while including the substituted part, are also included in the present invention.

In the case of the existing recombinant reporter system used to confirm the activity of caspase 3 or caspase 7, there was a drawback in that the length of the gene encoding the wild-type fluorescent protein used as a reporter is increased by adding the DEVDG sequence to the wild-type fluorescent protein or using a linker. In the case of the present invention, in order to minimize modification of the wild-type fluorescent protein, a position having a sequence similar to DEVDG was selected among the sequences of the wild-type fluorescent protein and replaced with DEVDG. Therefore, the fluorescent protein variant prepared by the method of the present invention has the advantages in that it has an amino acid sequence that is recognized and cleaved by caspase 3 or caspase 7, can maintain the length of the wild-type gene, and confirm the loss of the fluorescent protein by cleavage without the need for a separate nucleotide sequence or blocker/quencher, thus not requiring a complex structure.

The term “caspase” of the present invention is a kind of proteolytic enzyme that causes apoptosis by cleaving a specific site of a target protein. Specifically, caspases 8, 9, 10, etc. can act at the beginning of cell damage to activate the caspase cascade, and caspases 2, 3, 6, 7, etc. can eventually degrade DNA and proteins to cause apoptosis.

The “hinge region” refers to a region other than the α-helix and β-sheet regions that are considered to be related to the function of a fluorescent protein, and corresponds to, for example, a Bond outlier region. The “hinge region that does not affect the function of a fluorescent protein” refers to a part in which even a mutation in the amino acid sequence of the corresponding region does not significantly affect the function of the protein, and it can be understood that a loss of 40% or less of a fluorescent protein function due to a mutation does not have a significant effect.

In an embodiment of the present invention, when preparing a variant of a fluorescent protein, as the position to be mutated, the hinge region other than the part that affects the function of the fluorescent protein such as the α-helix and β-sheet of the fluorescent protein, was targeted.

In an embodiment of the present invention, part of the amino acid sequences in the hinge region may be substituted to include an amino acid sequence that is cleaved by a caspase.

This is advantageous in that by substituting the existing sequence in the hinge region, the wild-type gene length can be maintained, thereby minimizing the modification of the fluorescent protein and not requiring a separate nucleotide sequence or blocker/quencher.

The fluorescent protein variant may be one in which an asparagine-isoleucine-glutamic acid (NIE) sequence in the amino acid sequence of a fluorescent protein before mutation is substituted with an aspartic acid-glutamic acid-valine (DEV) sequence. In particular, the asparagine-isoleucine-glutamic acid (NIE) sequence may be the 171^{st} to 173^{rd} amino acids in the amino acid sequence represented by SEQ ID NO: 1, the 171^{st} to 173^{rd} amino acids in the amino acid sequence represented by SEQ ID NO: 33, and the 172^{nd} to 174^{th} amino acids in the amino acid sequence represented by SEQ ID NO: 34, or the 171^{st} to 173^{rd} amino acids in the amino acid sequence represented by SEQ ID NO: 35.

Therefore, the fluorescent protein variant may be one, in which asparagine (N), isoleucine (I), and glutamic acid (E), which are the 171^{st} to 173^{rd} amino acids in the amino acid sequence represented by SEQ ID NO: 1, are substituted with aspartic acid (D), glutamic acid (E), and valine (V), respectively.

In addition, the fluorescent protein variant may be one, in which asparagine (N), isoleucine (I), and glutamic acid (E), which are the 171^{st} to 173^{rd} amino acids in the amino acid sequence represented by SEQ ID NO: 33, are substituted with aspartic acid (D), glutamic acid (E), and valine (V), respectively.

In addition, the fluorescent protein variant may be one, in which asparagine (N), isoleucine (I), and glutamic acid (E), which are the 172^{nd} to 174^{th} amino acids in the amino acid sequence represented by SEQ ID NO: 34, are substituted with aspartic acid (D), glutamic acid (E), and valine (V), respectively.

In addition, the fluorescent protein variant may be one, in which asparagine (N), isoleucine (I), and glutamic acid (E), which are the 171^{st} to 173^{rd} amino acids in the amino acid sequence represented by SEQ ID NO: 35, are substituted with aspartic acid (D), glutamic acid (E), and valine (V), respectively.

In an embodiment of the present invention, a cell line expressing a fluorescent protein mutant is prepared in which a portion in the hinge region of a fluorescent protein is mutated into a DEVDG sequence cleaved by caspase 3 or 7, and it was confirmed that the cell line uniformly expresses fluorescence and that such fluorescence expression was significantly reduced by cell damage (Example 8). Meanwhile, in the case of a fluorescent protein variant prepared by inserting the DEVDG sequence between the C-terminal region of the fluorescent protein and the quenching peptide, it was predicted that cells expressing the fluorescent protein variant could exhibit fluorescence by cleavage due to cell damage (i.e., due to cleavage by caspase 3 or 7), but it was confirmed that fluorescence could be exhibited even without cell damage, and fluorescence expression did not appear uniformly in all cells after cell damage (Comparative Example 2). Therefore, compared to the existing method of mutating the DEVDG sequence in the C-terminal region of a fluorescent protein, mutating the hinge region of a fluorescent protein is more effective in detecting fluorescence expression caused by cell damage.

Another aspect of the present invention provides a polynucleotide encoding the fluorescent protein.

The polynucleotide may be a nucleotide sequence represented by SEQ ID NO: 14. Specifically, a nucleotide sequence that is at least 90%, 95%, 96%, 97%, 98%, 99%, or 100% identical to the nucleotide sequence represented by SEQ ID NO: 14 is also included in the present invention. In addition, the polynucleotide may be a nucleotide sequence represented by SEQ ID NO: 39. Specifically, a nucleotide sequence that is at least 90%, 95%, 96%, 97%, 98%, 99%, or 100% identical to the nucleotide sequence represented by SEQ ID NO: 39 is also included in the present invention. In addition, the polynucleotide may be a nucleotide sequence represented by SEQ ID NO: 40. Specifically, a nucleotide sequence that is at least 90%, 95%, 96%, 97%, 98%, 99%, or 100% identical to the nucleotide sequence represented by SEQ ID NO: 40 is also included in the present invention. In addition, the polynucleotide may be a nucleotide sequence represented by SEQ ID NO: 41. Specifically, a nucleotide sequence that is at least 90%, 95%, 96%, 97%, 98%, 99%, or 100% identical to the nucleotide sequence represented by SEQ ID NO: 41 is also included in the present invention.

Still another aspect of the present invention provides a recombinant vector including a polynucleotide.

The vector may be an adeno-associated virus integration site 1 (AAVS1) vector. The AAVS1 vector targets the integration of the insert DNA into AAVS1, a safe harbor of the human genome located on human chromosome 19. AAVS1-targeted gene introduction has been confirmed to be a safe site that does not affect the cell phenotype, and is known to enable stable and sustained expression of the transgene in many cell types, including embryonic stem cells. In an embodiment of the present invention, it was confirmed that a cell line exhibiting uniform fluorescence expression could be prepared using the AAVS1 SA-2A-puro-pA donor (#22075, Addgene) vector (Example 8). Meanwhile, it was confirmed that when using a vector that does not target the AAVS1 locus, it was difficult to establish a cell line exhibiting uniform fluorescence expression (Comparative Example 1). Therefore, in order to establish a cell line that exhibits uniform fluorescence expression, it is essential to use a vector that allows insertion of a gene encoding a fluorescent protein variant into the AAVS1 locus.

The recombinant vector may further include an antibiotic resistance gene. The antibiotic resistance gene may be a resistance gene for one or more antibiotics selected from the group consisting of penicillin, streptomycin, neomycin, puromycin, and hygromycin, and specifically, the antibiotic resistance gene may be a gene resistant to puromycin.

Still another aspect of the present invention provides a composition for detecting cell damage or apoptosis, which includes the fluorescent protein variant, polynucleotide, or recombinant vector as an active ingredient.

The composition may include a fluorescent protein variant, a polynucleotide, or a recombinant vector alone or may further include one or more pharmaceutically acceptable carriers.

Still another aspect of the present invention provides a transformed cell transformed with the recombinant vector.

As methods for transforming the recombinant vector into cells, methods known in the art, for example, transient transfection, microinjection, transduction, cell fusion, calcium phosphate precipitation, liposome-mediated transfection, DEAE dextran-mediated transfection, polybrene-mediated transfection, electroporation, gene gun, or other known methods for introducing nucleic acids into cells may be used, but are not limited thereto.

In addition, a transformed cell of hetero genotype in which the recombinant vector is inserted into one allele of a cell or a transformed cell of homo genotype into which both alleles are inserted may be constructed. In an embodiment of the present invention, AAVS1-EGFP #4 vector was introduced into HEK 293 cells, cultured, and primary selection was performed with puromycin, followed by secondary selection of cells expressing only GFP by FACS, and genotyping PCR was performed targeting the selected cells, and cells of wild-type, hetero genotype, and homo genotype were distinguished by comparing the band sizes of the samples, thereby preparing cells of hetero genotype or homo genotype into which the recombinant vector was introduced.

A method for confirming whether the recombinant vector has been introduced into the transformed cells may be a molecular biological assay or biochemical assay known in the art. Molecular bioassay methods include Southern and Northern blottings, RT-PCR and PCR, and biochemical assay methods include detection of the presence or absence of specific proteins/peptides by immunological methods (e.g., ELISA and Western blot). In an embodiment of the present invention, introduction of the vector into cells was confirmed by performing genotype PCR.

The method may further include isolating transformed cells expressing a fluorescent protein.

In the method of isolating the transformed cells expressing a fluorescent protein, the transformed cells expressing a fluorescent protein may be isolated through a process of first selecting antibiotic-resistant cells by treatment with an antibiotic. In addition, the method for isolating human cells expressing a fluorescent protein can isolate transformed cells expressing a fluorescent protein through a process of selecting cells expressing the fluorescent protein using fluorescence-activated cell sorting (FACS). Furthermore, the method of isolating human cells expressing a fluorescent protein includes first selecting for human cells resistant to antibiotics by treating them with antibiotics, and then selecting cells expressing a fluorescent protein using FACS.

In an embodiment of the present invention, after inoculating HEK 293 cells into a 100 mm culture dish, the cells were transfected with 5 μg of AAVS1-EGFP #4 vector, and 3 days thereafter, when the cells were cultured to about 90% confluence, puromycin selection was performed by treating 10 mL of the culture medium with 3 μL of puromycin (#A11138-03, gibco), and 14 days thereafter, when the cells were cultured to about 70% to 80% confluence, only the cells expressing only a green fluorescence protein were selected secondarily using FACS.

Still another aspect of the present invention provides a method for evaluating drug toxicity, which includes: i) treating a sample with transformed cells; ii) measuring fluorescence expression of cells treated with the sample; and iii) determining that the sample is toxic to cells when fluorescence expression changes. In particular, the transformed cells are the same as described above.

As the methods for measuring the fluorescence expression of the cells in step ii), biological assays known in the art, such as Southern and Northern blottings, RT-PCR and PCR; biochemical assays such as fluorescence microscopy, immunological methods (e.g., FACS, ELISA, and Western blot) may be used to confirm the measurements in real time. In an embodiment of the present invention, the expression level of the fluorescent protein was confirmed in real time using a fluorescence microscope and flow cytometry (FACS).

The change in fluorescence expression in step iii) may be due to the activity of caspase 3 or caspase 7.

In the drug toxicity evaluation method in step iii), when the expression level of the fluorescent protein is measured by treating the sample with the transformed cells, if the expression level of the fluorescent protein decreases, it may be determined that the sample is toxic to the cells.

Hereinafter, the present invention will be described in more detail by the following examples. However, the following examples are only for illustrating the present invention, and the scope of the present invention is not limited thereto.

### EMBODIMENTS FOR CARRYING OUT THE INVENTION

### I. Preparation of Green Fluorescent Protein Variant Vector System

In order to monitor the expression of green fluorescent protein in real time by the activity of caspase 3 or caspase 7 in cells, part of the amino acid sequence of the hinge region that does not affect the function of green fluorescent protein was subjected to DEVDG mutation, and thereby, an AAVS1 vector including the gene encoding the green fluorescent protein variant was prepared.

### Example 1. Selection of Target Amino Acid Candidates in Green Fluorescent Protein for Preparation of Green Fluorescent Protein Variants

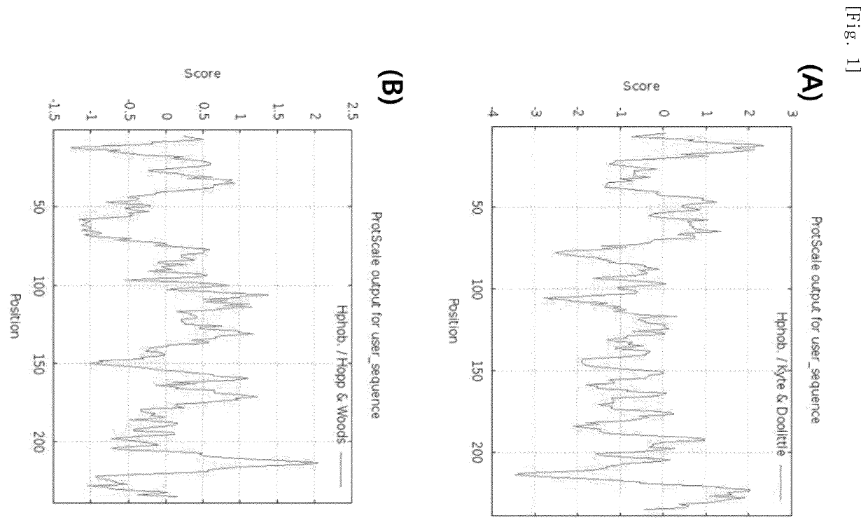

In order to predict the hydropathic amino acid portion that can bind to a drug in the amino acid sequences (SEQ ID NO: 1) of enhanced green fluorescent protein (EGFP), the amino acid sequence of the EGFP gene (SEQ ID NO: 2) used in the present invention (MVSKGEELFTGVVPILVELDGDVNGHKFSVSGEGEGDATYGKLTLKFICTTG KLPVPWPTLVTTLTYGVQCFSRYPDHMKQHDFFKSAMPEGYVQERTIFFKDD GNYKTRAEVKFEGDTLVNRIELKGIDFKEDGNILGHKLEYNYNSHNVYIMADK QKNGIKVNFKIRHNIEDGSVQLADHYQQNTPIGDGPVLLPDNHYLSTQSALSK DPNEKRDHMVLLEFVTAAGITLGMDELYK) was input into the ExPASy ProtScale site (https://web.expasy.org/protscale/), and the amino acid sequence was analyzed using the Kyte-Doolittle scale and the Hopp-Woods scale (FIG. 1). In particular, a positive number on the Kyte-Doolittle scale means hydrophobicity, a negative number indicates hydrophilicity, and a positive number on the Hopp-Woods scale means hydrophilicity.

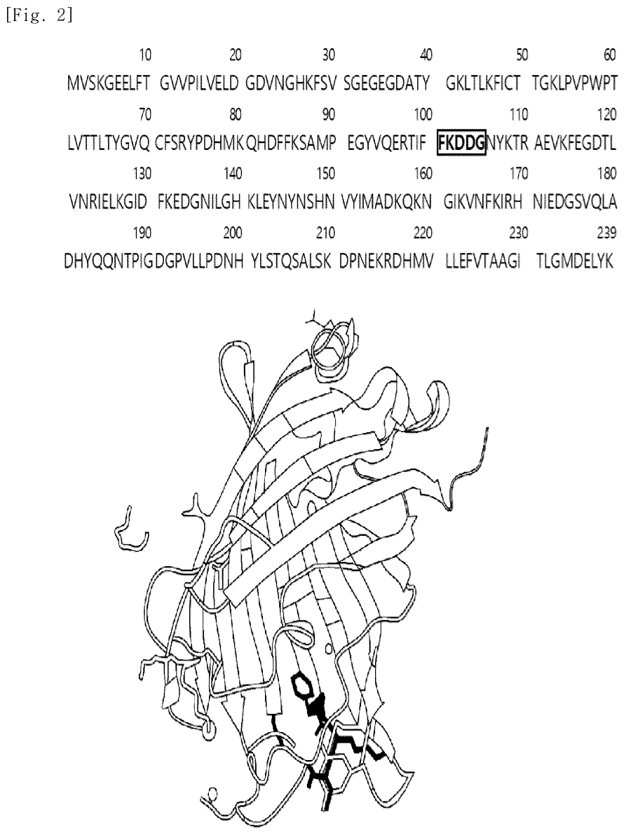

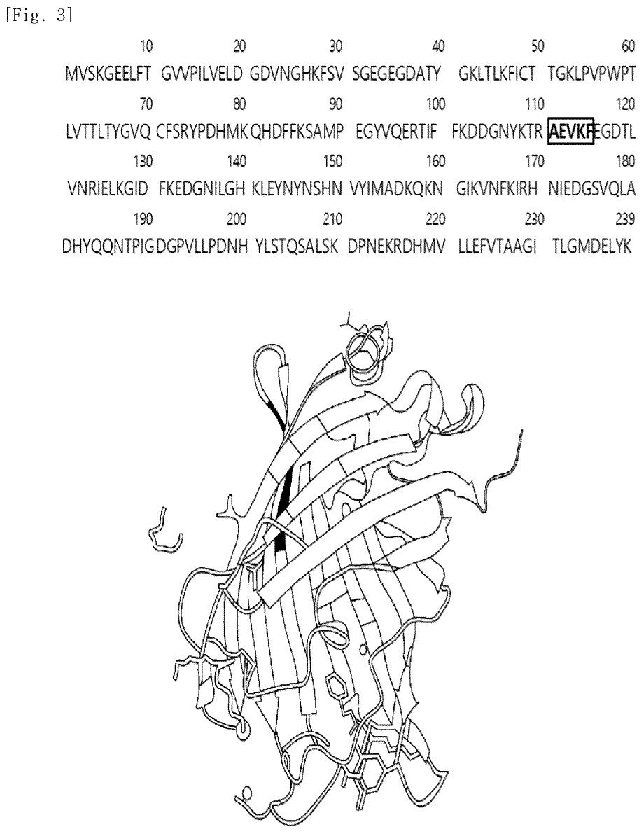

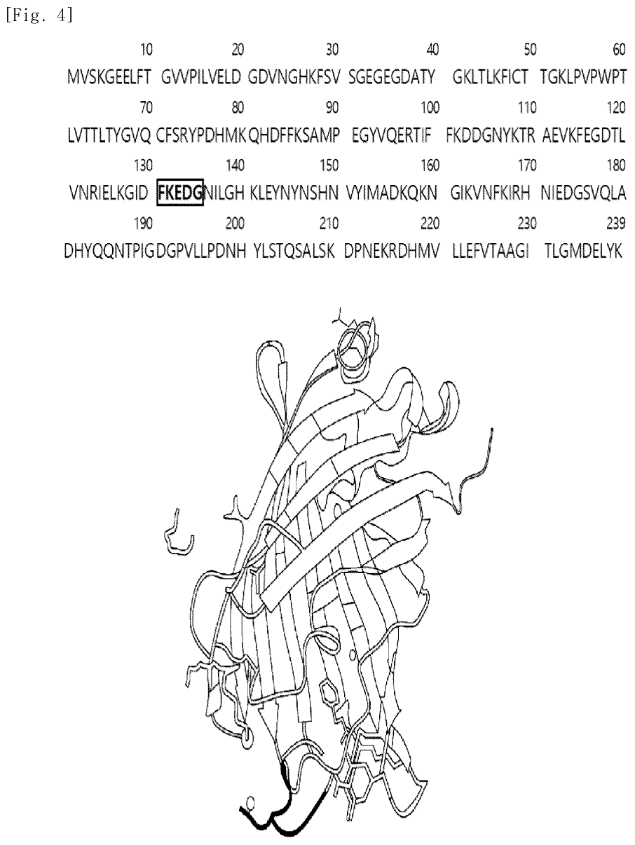

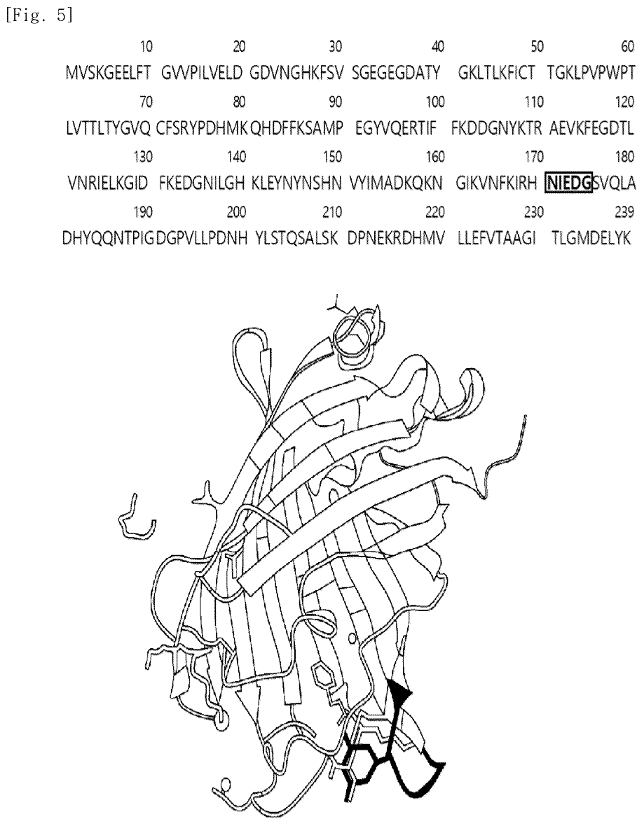

In order to select an amino acid sequence that can be mutated to DEVDG in the amino acid region predicted to be hydrophilic through the process above, each of hydrophilic amino acid regions was analyzed using the information on the RCSB PDB site (https://www.rcsb.org/structure/4EUL). Specifically, among the hydrophilic amino acid regions, the Bond outlier region that does not affect the properties and structure of a green fluorescent protein, such as hydrophilicity, polarity, buriedness, accessibility, rotatability, flexibility, and bendability, was selected. The Bond outlier region is the hinge region, which is a region other than the α-helix and β-sheet regions of the protein structure, and is understood to not affect the function of the protein. In particular, in order to minimize mutations in the wild-type enhanced green fluorescent protein (EGFP WT), the region with an amino acid sequence similar to DEVDG was selected as a target. The amino acid sequences of the selected target candidate sequences #1 to #4 and the positions of the target candidate sequences #1 to #4 on the three-dimensional structure of EGFP are indicated by the sequences in the boxes and the black areas in FIGS. 2 to 5, respectively.

### Example 2. DEVDG Mutation of Target Amino Acid

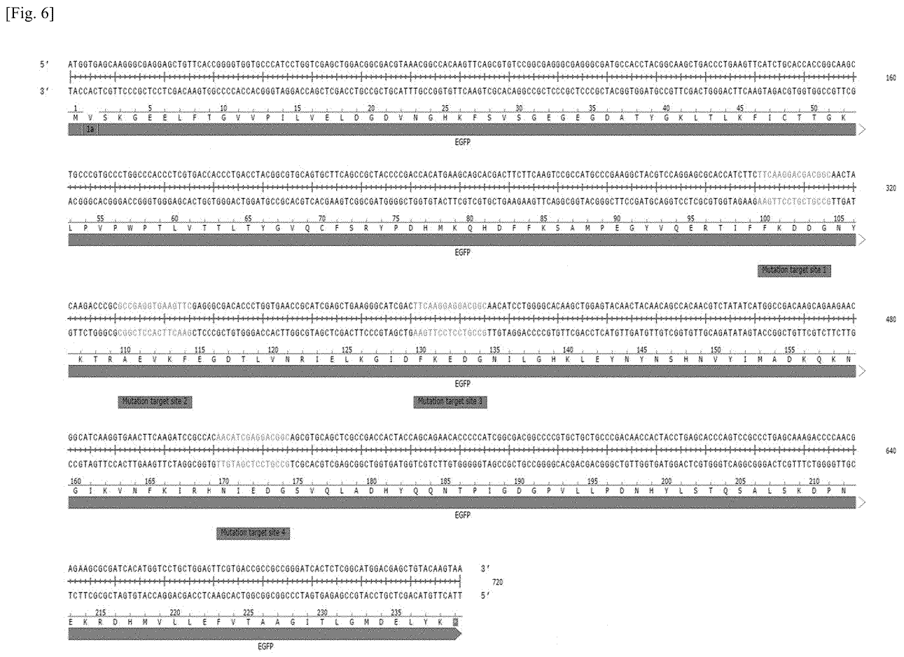

The positions on the nucleotide sequence of EGFP for the target candidates #1, #2, #3, and #4 selected through Example 1 are indicated in FIG. 6.

With regard to the nucleotide sequences of the EGFP target candidates #1 to #4 (Table 1), the nucleotide sequence GACGAGGTCGACGGC (SEQ ID NO: 11) for target candidate #1 was mutated using the Bioneer gene synthesis service, and target #2 to #4 were mutated to the nucleotide sequence GACGAGGTGGACGGC (SEQ ID NO: 12) so that all amino acid sequences encoded by this nucleotide sequence could be DEVDG (SEQ ID NO: 13). Among them, the full nucleotide sequence of the EGFP #4 variant is represented by SEQ ID NO: 14.

TABLE 1

| Target Candidate Groups | | | SEQ ID NO: |
|---|---|---|---|
| #1 | nucleotide sequence | TTCAAGGACGACGGC | 3 |
| | amino acid sequence | FKDDG | 4 |
| #2 | nucleotide sequence | GCCGAGGTGAAGTTC | 5 |
| | amino acid sequence | AEVKF | 6 |
| #3 | nucleotide sequence | TTCAAGGAGGACGGC | 7 |
| | amino acid sequence | FKEDG | 8 |
| #4 | nucleotide sequence | AACATCGAGGACGGC | 9 |
| | amino acid sequence | NIEDG | 10 |

### Example 3. Construction of pCAGGS Vector Including Genes Encoding Green Fluorescent Protein Variants

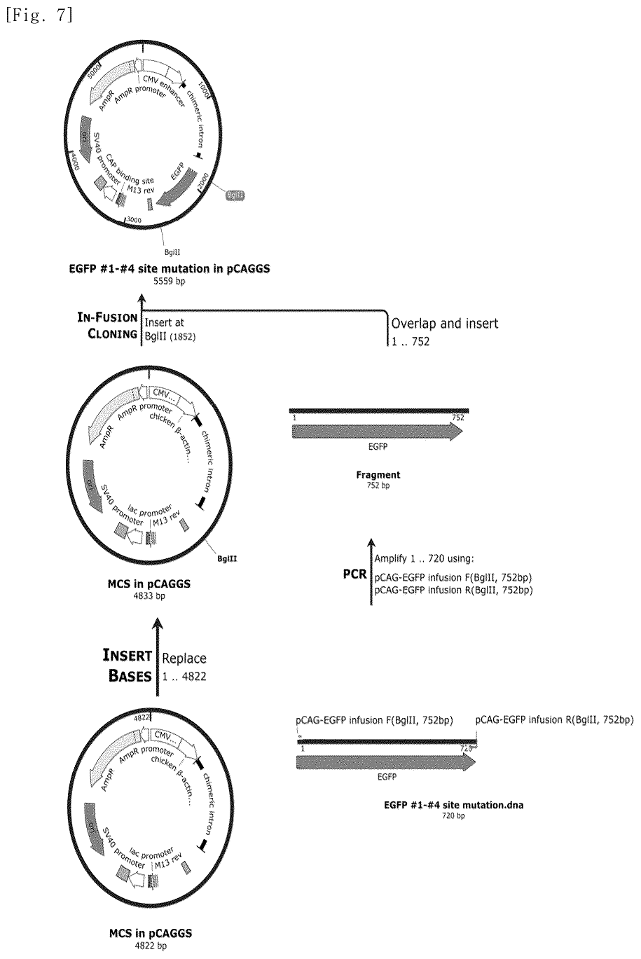

Enhanced green fluorescent protein mutant genes obtained by mutating target candidate sequences #1 to #4 of wild-type enhanced green fluorescent protein (EGFP), respectively, were cloned into the pCAGGS vector (FIG. 7).

### 3-1. Preparation of Insert DNA

In-Fusion PCR was performed under the conditions shown in Table 2 below using TaKaRa Ex Taq™ (#HRR001A, TAKARA) using wild-type EGFP and EGFP #1 to #4 site mutation genes as templates. In particular, the primers used for In-Fusion PCR are shown in Table 3 below. Wild-type EGFP and EGFP #1 to #4 mutant genes were engineered to have a BglII restriction enzyme site using the primers shown in Table 3.

TABLE 2

| In-Fusion PCR | Temperature | Hour |
|---|---|---|
| Step 1 | 98° C. | 2 min |
| Step 2: | 98° C. | 10 sec |
| 35 cycles | 62° C. | 10 sec |
| | 72° C. | 10 sec |
| Step 3 | 72° C. | 5 min |

Step 1: initial denaturation,
Step 2: amplification,
Step 3: final elongation

TABLE 3

| | | | SEQ ID |
|---|---|---|---|
| Primers | bp | Sequence | NO: |
| pCAG-EGFP | 752 bp | AATACCACTGAGATCTATGGTGAGCAA | 15 |
| infusion F | | GGGCGAGG | |
| pCAG-EGFP | 752 bp | AGAGGGAAAAAGATCTTTACTTGTACA | 16 |
| infusion R | | GCTCGTCCATGCC | |

Electrophoresis was performed on the amplified PCR product, and after recovering a band corresponding to the target size (752 bp) among the loaded samples through electrophoresis, DNA was extracted using the Geneclean® turbo kit (#111102-600, MP),

### 3-2. Preparation of Vector

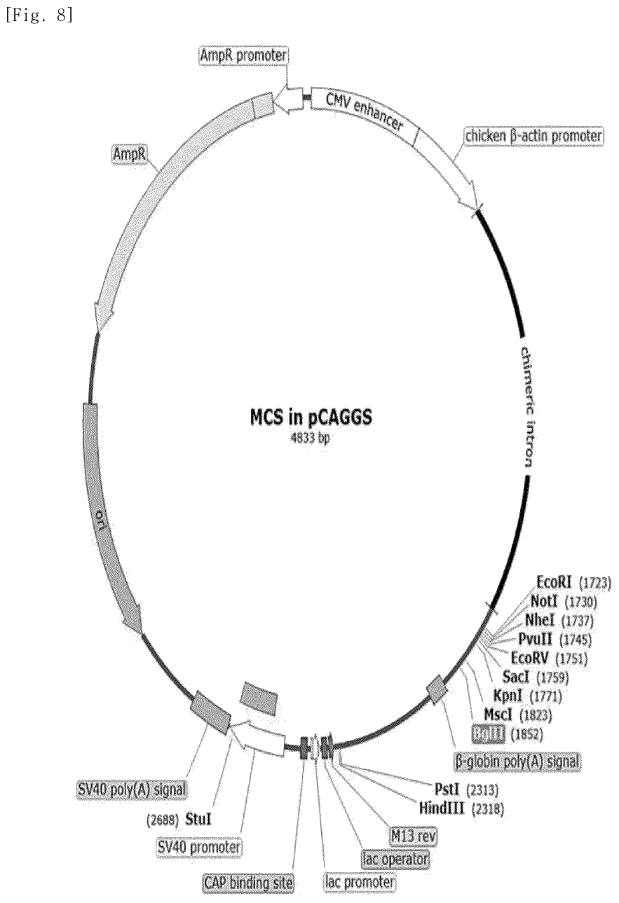

A BglII site was created in the multiple cloning site (MCS) of the pCAGGS vector, and when inserting the insert DNA, it was digested with a BglII (#R0144L, NEB) restriction enzyme and used as a backbone vector (FIG. 8).

### 3-3. Preparation of Wild-Type EGFP and EGFP #1 to #4 Site Mutated Expression Vectors by Ligation of Insert DNA to a Vector

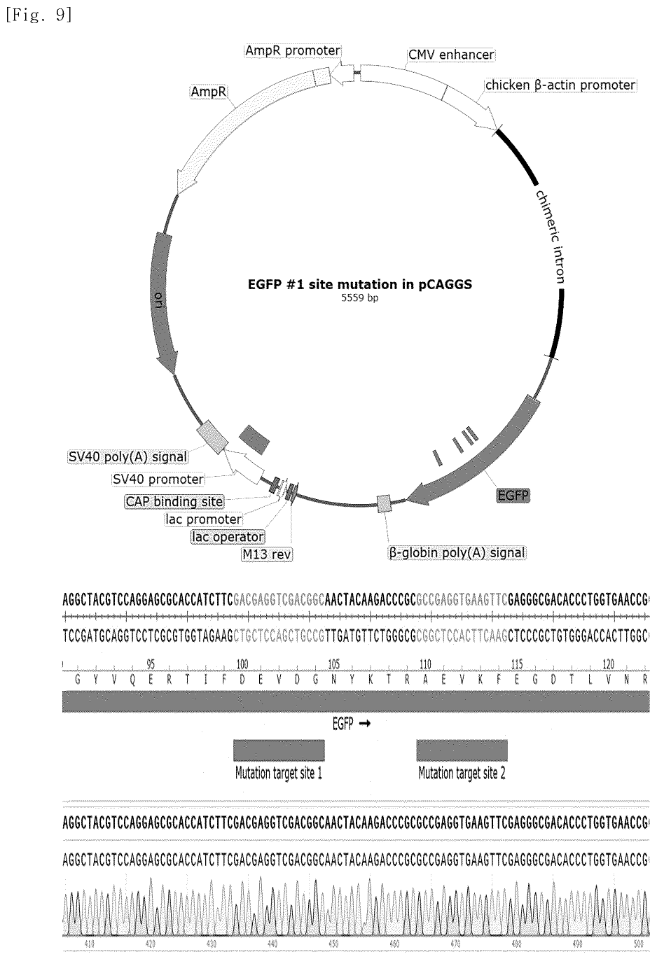

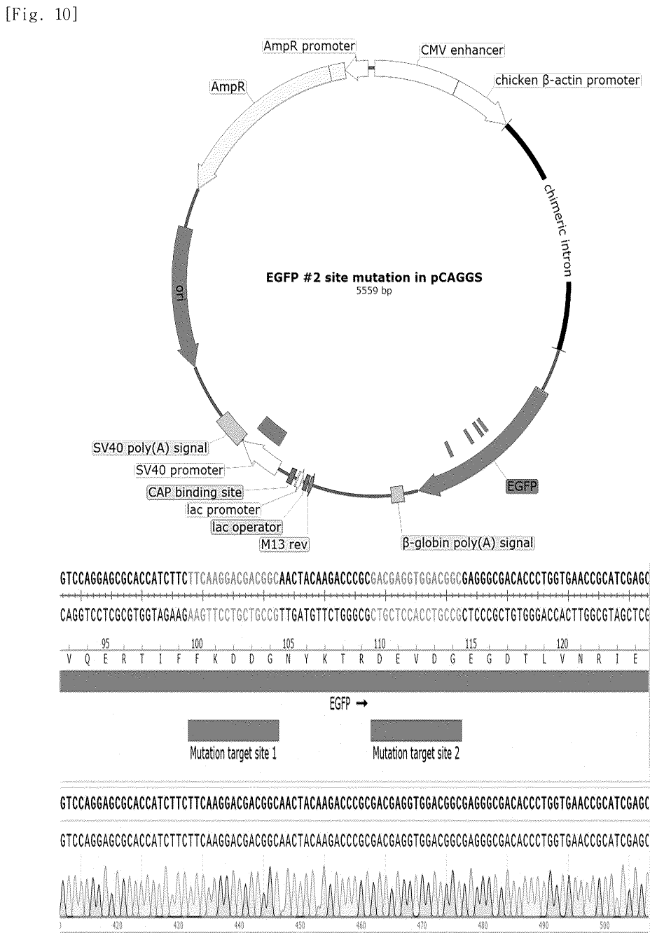

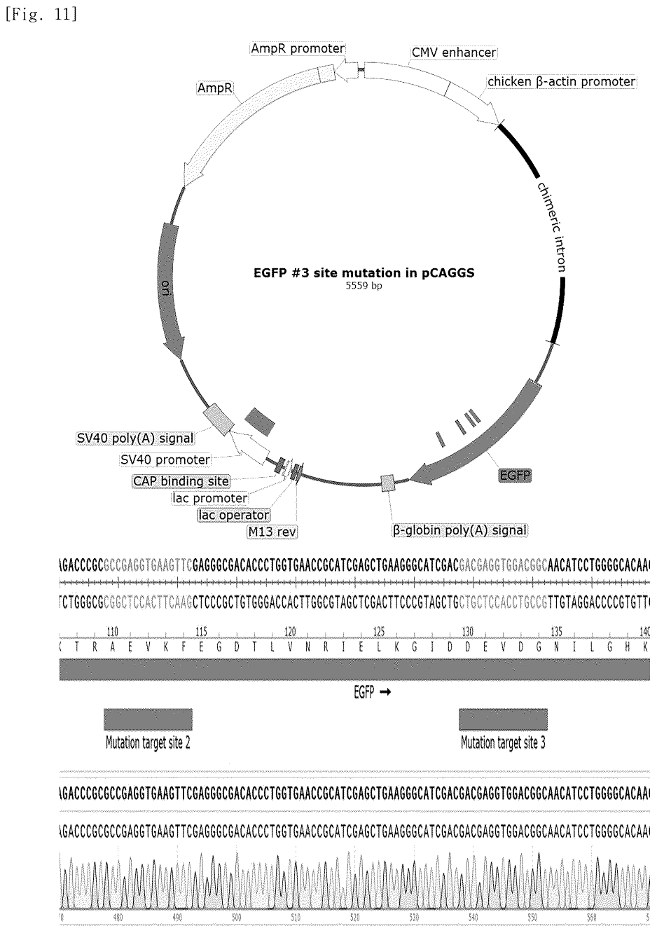

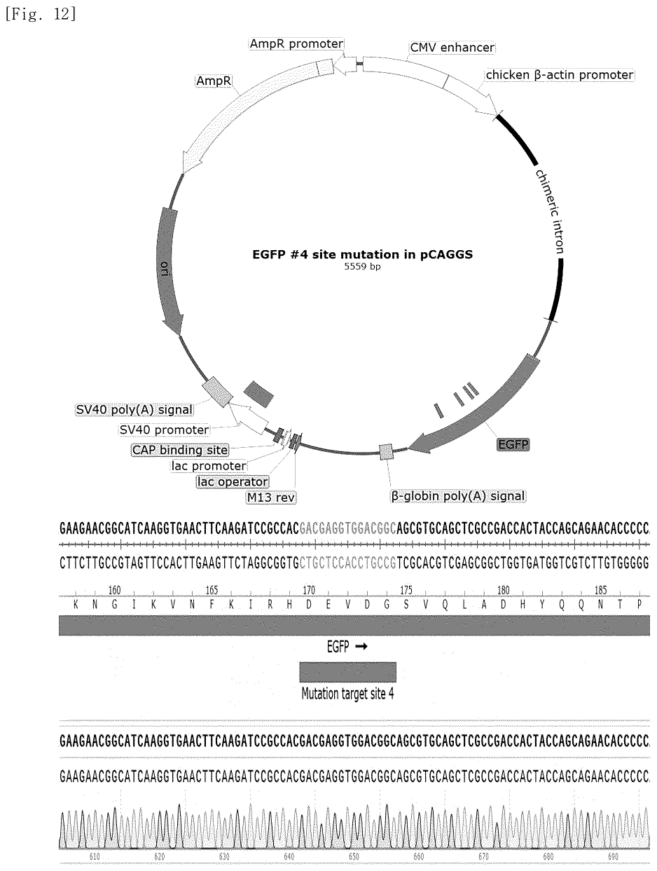

The insert DNA prepared in Example 3-1 was ligated to the vector prepared in Example 3-2 using the In-Fusion® HD Cloning Kit (#639649, TAKARA), and wild-type EGFP and EGFP #1 to #4 site mutated expression vectors were prepared. The pCAGGS vectors prepared separately for each insert are shown in FIGS. 9 to 12.

### 3-4. Transformation

The pCAGGS vector into which the genes of the wild-type EGFP and variants were inserted, respectively, was transformed into HIT Competent Cells-DH5alpha High 108 (#RH618, RBC) on an LB broth plate.

### Example 4. Verification of Fluorescence Expression by Target Sequence

The expression of the green fluorescent protein variant of each pCAGGS vector prepared in Example 3 was verified.

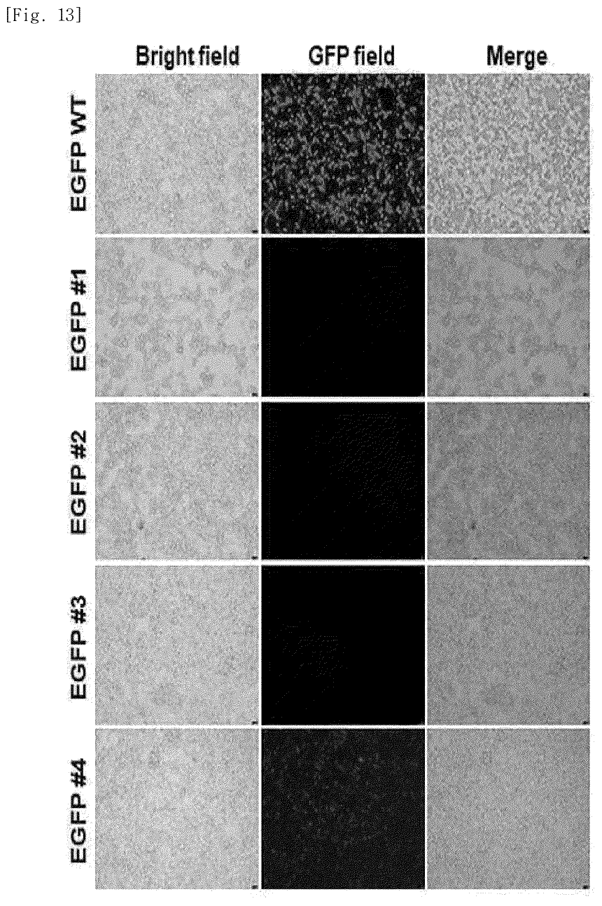

As a result of confirming through a fluorescence microscope the expression of the green fluorescent protein variants in the cell lines into which each pCAGGS vector was introduced, it was confirmed that the gene-mutated green fluorescent protein was normally expressed in the cell line into which the pCAGGS vector including the EGFP #4 mutant gene was introduced, and it was confirmed that the green fluorescent protein variant was hardly expressed in other cell lines into which the pCAGGS vector was introduced (FIG. 13).

### Example 5. Construction of AAVS1 Vectors Including Genes Encoding Green Fluorescent Protein Variants

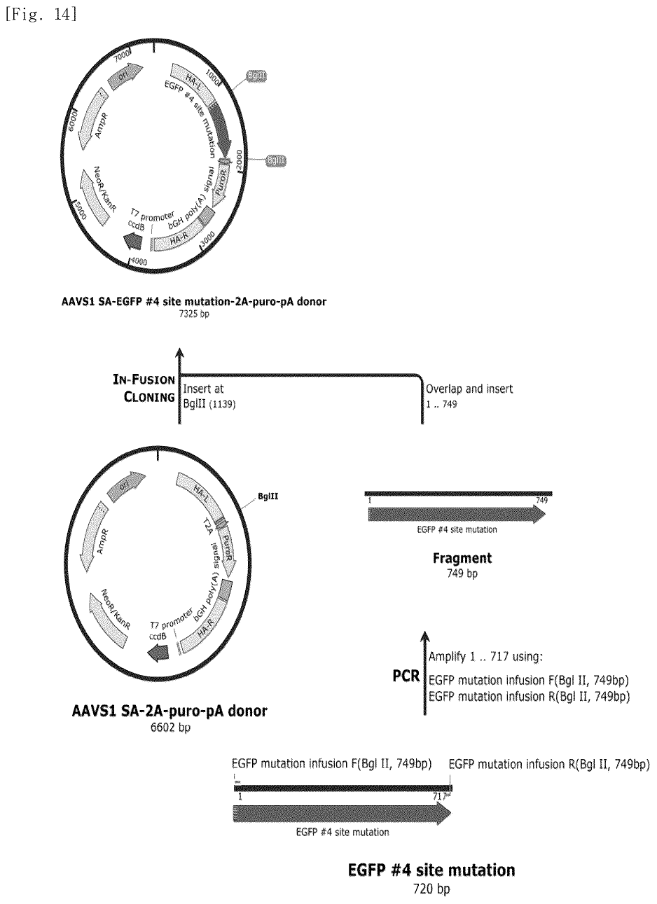

The EGFP #4 variant gene verified in Example 4 was cloned into the AAVS1 SA-2A-puro-pA donor vector (#22075, Addgene) (FIG. 14).

### 5-1. Preparation of Insert DNA

In-Fusion PCR was performed under the conditions shown in Table 4 below using TaKaRa Ex Taq™ (#HRR001A, TAKARA) with the EGFP #4 variant gene as a template. In particular, the primers used for In-Fusion PCR are shown in Table 5 below. A BglII restriction enzyme site was prepared in the EGFP #4 variant gene using the primers shown in Table 5.

TABLE 4

| In-Fusion PCR | Temperature | Hour |
|---|---|---|
| Step 1 | 98° C. | 2 min |
| Step 2: | 98° C. | 10 sec |
| 35 cycles | 62° C. | 10 sec |
| | 78° C. | 10 sec |
| Step 3 | 72° C. | 5 min |

Step 1: initial denaturation,
Step 2: amplification,
Step 3: final elongation

TABLE 5

| | | | SEQ ID |
|---|---|---|---|
| Primers | bp | Sequence | NO: |
| EGFP mutation | 749 bp | GGGCCTCGAGAGATCTATGGTGAGCAA | 17 |
| infusion F | | GGGCGAGG | |
| EGFP mutation | 749 bp | CTCCGCTGCCAGATCTCTTGTACAGCT | 18 |
| infusion R | | CGTCCATGCC | |

Electrophoresis was performed on the amplified PCR product, and after recovering a band corresponding to the target size (749 bp) from the loaded sample through electrophoresis, DNA was extracted using the Geneclean® turbo kit.

### 5-2. Preparation of EGFP #4 Variant Expression Vector by Ligation of Insert DNA to a Vector

The extracted DNA fragment of 749 bp was used as insert DNA, and the AAVS1 SA-2A-puro-pA donor vector was digested with Bg/II restriction enzyme and used as a backbone vector. In particular, the ligation of the insert DNA and the backbone vector was performed using the In-Fusion® HD Cloning Kit. The prepared AAVS1 vector was named AAVS1 SA-EGFP #4 site mutation-2A-puro-pA donor (hereinafter “AAVS1-EGFP #4 vector”).

### 5-3. Transformation

In an LB broth plate, the HIT Competent Cells-DH5alpha High 108 (#RH618, RBC) cell line was transformed with the AAVS1-EGFP #4 vector prepared in Example 5-2 above.

### Example 6. Construction of Transformed Cell Lines Introduced with AAVS1 SA-EGFP #4 Vector

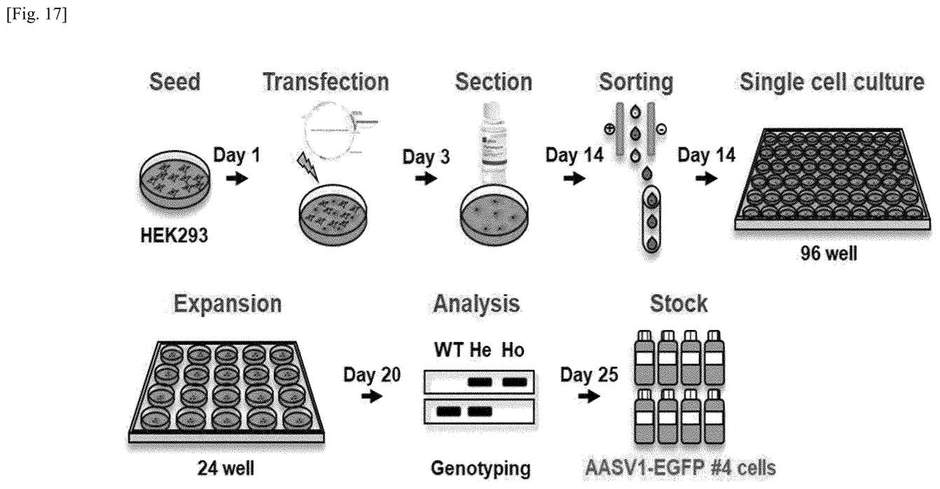

A transformed cell line was constructed by introducing the AAVS1 SA-EGFP #4 vector prepared in Example 5 into HEK 293 cells derived from human embryonic kidney (FIG. 17).

First, HEK 293 cells were inoculated into a 100 mm culture dish, and when the cells are cultured to about 70% to 80% of confluence, 5 μg of the AAVS1-EGFP #4 vector was introduced into the cells according to the SF kit, CM-130 program, and the manufacturer's manual using the 4D-Nucleofector™ equipment. After 3 days, when the cells were cultured to about 90% of confluence, puromycin selection was performed by adding 3 μL of puromycin (#A11138-03, gibco) to 10 mL of the culture medium. After 14 days, when the cells are cultured to about 70% to 80% of confluence again, cells expressing green fluorescent protein were secondarily sorted using fluorescence-activated cell sorting (FACS), and then the sorted single cells were seeded into 96-well plate, respectively. Transformed cell lines were constructed by inoculating and culturing in a 96-well plate and then subculturing in a 24-well plate.

The transformed cell line constructed through the above process was named AAVS1-EGFP #4 (DEVDG) 293 cell line, and through the same process as above, a cell line in which the AAVS1-EGFP #4 vector was inserted into one allele and a Cell line into which the AAVS1-EGFP #4 vector was inserted into both alleles were constructed.

### Example 7. Genotyping of Transformed Cell Lines

In order to confirm whether the AAVS1-EGFP #4 vector was correctly inserted into HEK 293 cells, genomic DNA was extracted from the cells in which EGFP fluorescence was expressed and maintained under a fluorescence microscope among the transformed cell lines obtained in Example 6. Then, genotyping PCR was performed to determine whether the gene was inserted.

Specifically, genomic DNA was extracted from cells for genotyping PCR using the DNeasy Blood & Tissue kit (#69506, Qiagen) according to the manufacturer's manual. Genotyping PCR was performed under the conditions shown in Table 6 above using the extracted genomic DNA and ExPrime Taq Premix (2×, 8-strip) (#G-6000, GeNet Bio).

TABLE 6

| In-Fusion PCR | Temperature | Hour |
|---|---|---|
| Step 1 | 95° C. | 5 min |
| Step 2: | 95° C. | 30 sec |
| 35 cycles | 62° C. | 30 sec |
| | 72° C. | 30 sec |
| Step 3 | 72° C. | 5 min |

Step 1: initial denaturation,
Step 2: amplification,
Step 3: final elongation

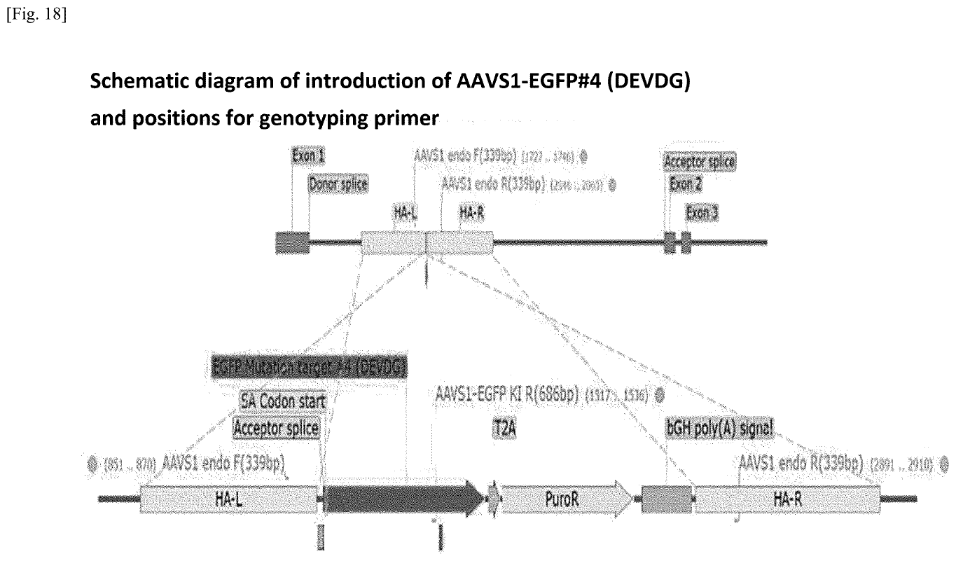

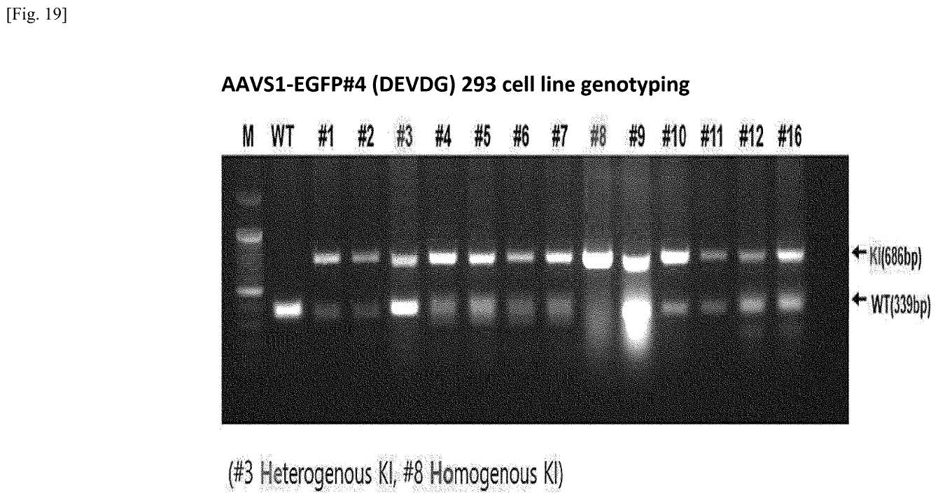

The samples subjected to genotyping PCR were electrophoresed on a 0.8% agarose gel prepared using agarose, LE, analytical grade (#V3125, Promega) and 0.5×TAE (50×TAE; TR2002-100-00, diluted to 0.5× using Biosesang 3^{rd} order) at 120 V for 30 minutes. Thereafter, by comparing the band sizes of the samples, wild-type, hetero-genotype, and homo-genotype cells were distinguished. PCR primers for amplification of genomic DNA were designed in silico using Primer3 software (https://bioinfo.ut.ee/primer3-0.4.0/), and are shown in FIG. 18 and Table 7 below. FIG. 19 shows the results of electrophoresis, and #1 to #16 in FIG. 19 show the cells in which EGFP fluorescence was expressed and maintained observed through a fluorescence microscope among the transformed cell lines obtained in Example 6.

TABLE 7

| Primers | bp | Sequence | SEQ ID NO: |
|---|---|---|---|
| AAVS1 endo F | 339 bp | CCCCTATGTCCACTTCAGGA | 19 |
| AAVS1 endo R | 339 bp | AGGATCCTCTCTGGCTCCAT | 20 |
| AAVS1-EGFP KI R | 686 bp | GTTCACCTTGATGCCGTTCT | 21 |

As a result, as shown in FIG. 19, in the case of HEK293 cells (WT) into which the AAVS1-EGFP #4 vector was not inserted (knock-in; KI), a PCR product of 339 bp appeared, in the case of hetero genotypes in which the vector was inserted into one allele (#2 to #7 and #9 to #16), 339 bp and 686 bp PCR products were observed, and in the case of the homo genotype (#8), only a 686 bp PCR product was observed.

### Example 8. Confirmation of Fluorescence Expression of Transformed Cell Lines

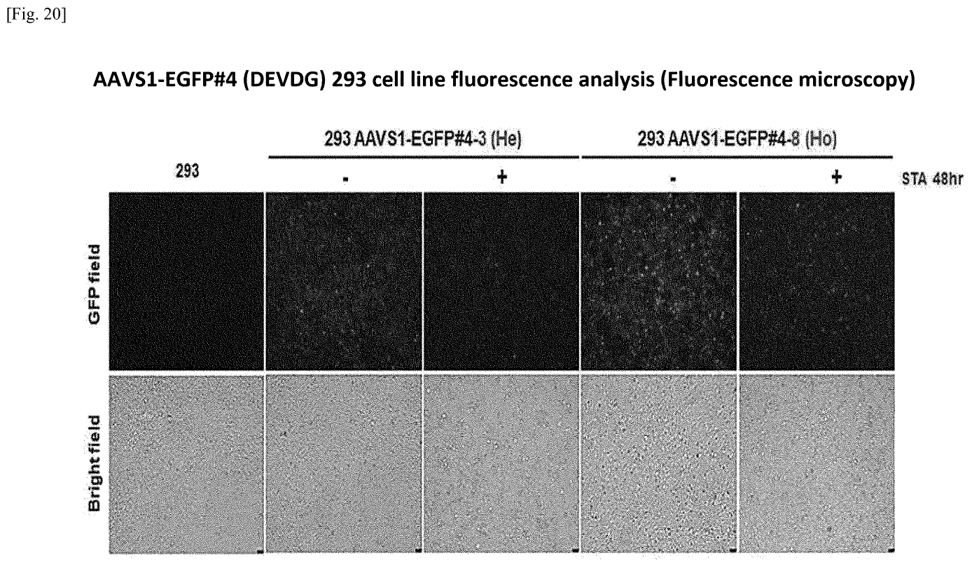

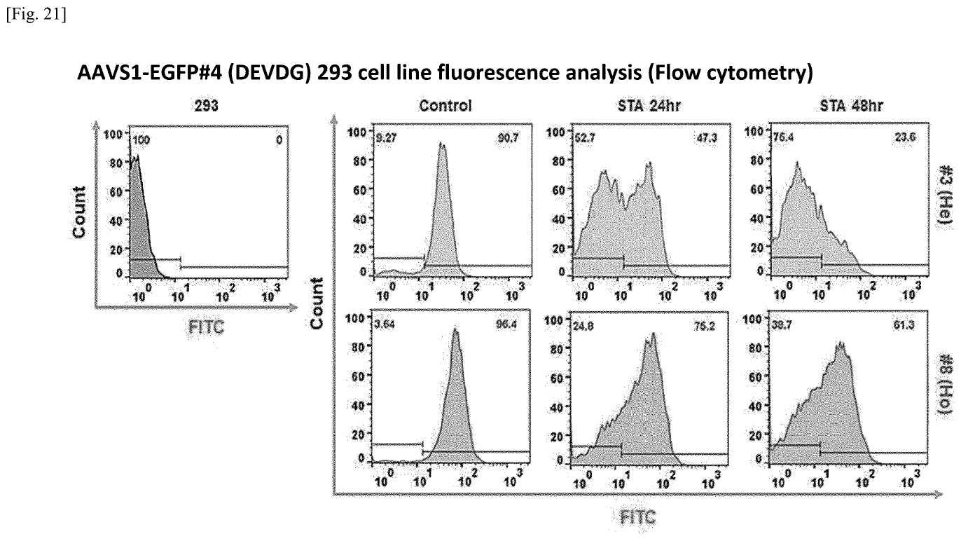

Among the HEK293 cells into which AAVS1-EGFP #4 vector was inserted and verified in Example 7, the hetero-genotype #3 cells and the homo-genotype #8 cells were each treated with staurosporine (STA), a substance that induces apoptosis, for 48 hours, and confirmed through fluorescence microscopy and flow cytometry, the change in fluorescence, which are shown in FIGS. 20 and 21, respectively.

As a result, it was confirmed through fluorescence microscopy that fluorescence decreased in both the hetero genotype, in which the vector was inserted into one allele, and the homo genotype, in which the vector was inserted into both alleles, when treated with staurosporine at a concentration of 1 μM for 48 hours. In addition, it was confirmed that fluorescence was strongly expressed in the homo genotype, in which the vector was inserted into both alleles, compared to the hetero genotype, in which the vector was inserted into one allele (FIG. 20).

In addition, as shown in FIG. 21, it was confirmed that the fluorescence decreased in real time when treated for 48 hours, as observed through flow cytometry, compared to when staurosporine was treated for 24 hours in both the hetero genotype, in which the vector was inserted into one allele, and the homo genotype, in which the vector was inserted into both alleles. In addition, it was confirmed that changes in real-time fluorescence were better observed in the homo genotype, in which the vector was inserted into both alleles compared to the hetero genotype, in which the vector was inserted into one allele.

Through this, it was confirmed that stronger fluorescence expression could be induced by inserting the AAVS1-EGFP #4 vector into both alleles, and changes in fluorescence due to apoptosis were observed in real time through HEK293 cells into which the AAVS1-EGFP #4 vector was inserted.

### Example 9. Confirmation of mRNA Expression Level of Transformed Cell Line

The HEK293 cells, into which AAVS1-EGFP #4 vector was inserted and verified in Example 7, were treated with staurosporine, a substance that induces apoptosis, for 48 hours, and the mRNA expression level of the green fluorescent protein variant was measured by semiquantitative RT-PCR (semi-RT-PCR).

Specifically, HEK293 wild-type cells (WT) without the insertion of AAVS1-EGFP #4 vector, #3 cells of AAVS1-EGFP #4 hetero genotype and #8 cells of the EGFP #4 homo genotype, among the AAVS1-EGFP #4 vector-inserted HEK293 cells verified in Example 7, were treated with staurosporine for 48 hours.

Thereafter, total RNA was extracted from the cells using RNeasy plus mini kit (#74136, Qiagen) according to the manufacturer's manual. The extracted total RNA (1 μg) was synthesized into cDNA using the PrimeScript RT reagent kit with gDNA Eraser (#RR047A, Takara Bio Inc) according to the manufacturer's manual. Genotyping PCR was performed using the cDNA as a template using ExPrime Taq Premix (2×, 8-strip) (#G-6000, GeNet Bio) under the conditions shown in Table 8 below.

TABLE 8

| Semi-RT PCR | Temperature | Hour |
|---|---|---|
| Step 1 | 95° C. | 5 min |
| Step 2: | 95° C. | 30 sec |
| 35 cycles | 62° C. | 30 sec |
| | 72° C. | 30 sec |
| Step 3 | 72° C. | 5 min |

Step 1: initial denaturation,
Step 2: amplification,
Step 3: final elongation

The samples subjected to genotyping PCR were electrophoresed on a 1.0% agarose gel prepared using agarose, LE, analytical grade (#V3125, Promega) and 0.5×TAE (50×TAE; TR2002-100-00, diluted to 0.5× using Biosesang 3rd order) at 120 V for 30 minutes. Thereafter, the bands of the samples were compared to confirm the level of mRNA expression. PCR primers for amplification of genomic DNA were designed in silico using Primer3 software (https://bioinfo.ut.ee/primer3-0.4.0/) and are shown in Table 9 below.

TABLE 9

| | | | SEQ ID |
|---|---|---|---|
| Primers | bp | Sequence | NO: |
| EGFP semi-RT PCR F | 245 bp | AGAAGAACGGCATCAAGGTG | 22 |
| EGFP semi-RT PCR R | 245 bp | CTTGTACAGCTCGTCCATGC | 23 |
| GAPDH semi-RT PCR F | 131 bp | GAGCCACATCGCTCAGACAC | 24 |
| GAPDH semi-RT PCR R | 131 bp | AAGGGGTCATTGATGGCAAC | 25 |

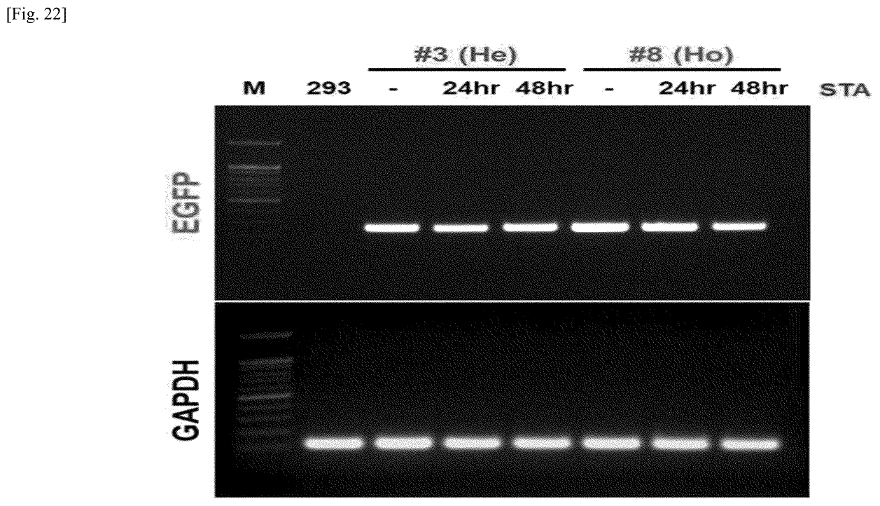

As a result, it was confirmed through semi RT-PCR that when staurosporine was treated for 24 hours and 48 hours, the fluorescence decreased in real time, whereas the expression level of the green fluorescent protein variant mRNA did not change (FIG. 22).

Through this, it was confirmed that the changes in fluorescence in HEK293 cells inserted with the AAVS1-EGFP #4 vector was caused by the cleavage of the DEVDG sequence of the green fluorescent protein variant.

### Example 10. Measurement of Cell Damage of Transformed Cell Lines and Confirmation of Levels of Fluorescence Expression and Expression of Cell Damage-Related Proteins

#8 Cells of AAVS1-EGFP #4 homo genotype described in Example 7 were treated with staurosporine, a substance that induces apoptosis, at concentrations of 0.1 μM, 0.25 μM, 0.5 μM, and 1 μM, respectively, for 48 hours, and then, the fluorescence expression of green fluorescent protein variants was confirmed by fluorescence microscopy and flow cytometry, and the expression levels of cell damage-related proteins such as Caspase 3, Cleaved Caspase 3, Cytochrome C, PARP, and Cleaved PARP were confirmed by Western blot.

Specifically, Western blot was performed through the following process. After separating 30 μg to 60 μg of protein lysates on 8% to 12% sodium dodecyl sulfate-polyacrylamide gel through SDS-PAGE, they were transferred to 0.45 μm nitrocellulose membranes (Cat #HAWP04700, EMD Millipore, Burlington, MA, USA).

The membranes were immersed into a solution containing 10 mM Tris pH 7.4, 150 mM NaCl, and 0.02% Tween-20 (TBST), and then blocked with 5% skim milk (Cat #232100, BD Biosciences) or bovine serum albumin (Cat #A9647, Sigma-Aldrich), and incubated overnight at 4° C. along with primary antibodies (GFP, Cat #50430-2-AP, Proteintech, 1:2000; Caspase 3, Cat #9662S, Cell signaling technology, 1:1000; Cleaved Caspase-3, Cat #9664L, Cell signaling technology, 1:1000; Cytochrome C, Cat #4280S, Cell signaling technology, 1:1000; PARP, Cat #9532S, Cell signaling technology, 1:1000; Cleaved PARP, Cat #5625S, Cell signaling technology, 1:1000; GAPDH, Cat #LF-MA0038, AB Frontier, 1:2000).

Thereafter, the membrane was washed three times with TBST, blocked again with 5% skim milk, and incubated with horseradish peroxidase-conjugated secondary antibody at room temperature for 1 hour. After the incubation, blots were washed with TBST and antibody binding was confirmed using SuperSignal West Pico PLUS Chemiluminescent Substrate (Cat #34580, Thermo Fisher Scientific) according to the manufacturer's instructions.

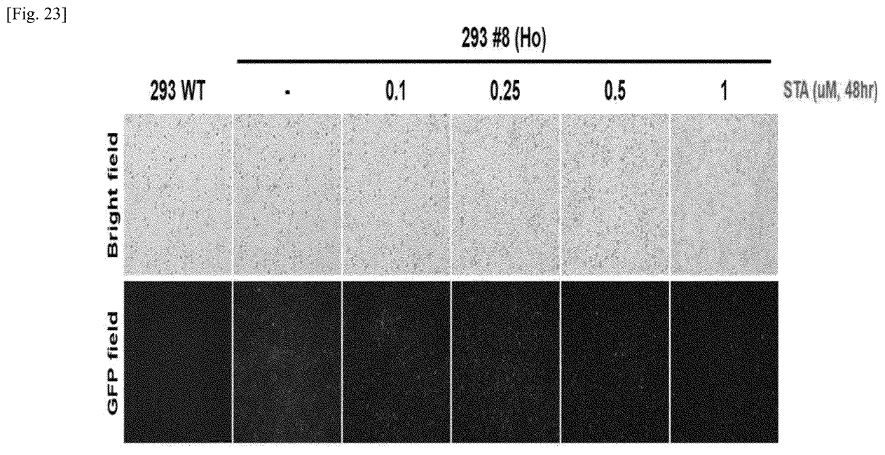

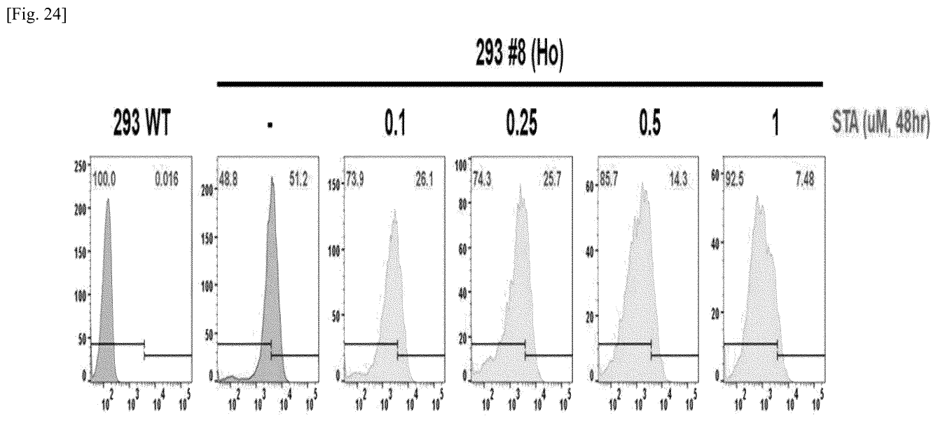

As a result, it was confirmed that the expression of green fluorescent protein decreased in #8 cells of AAVS1-EGFP #4 homo genotype as the treatment concentration of staurosporine increased (FIGS. 23 and 24).

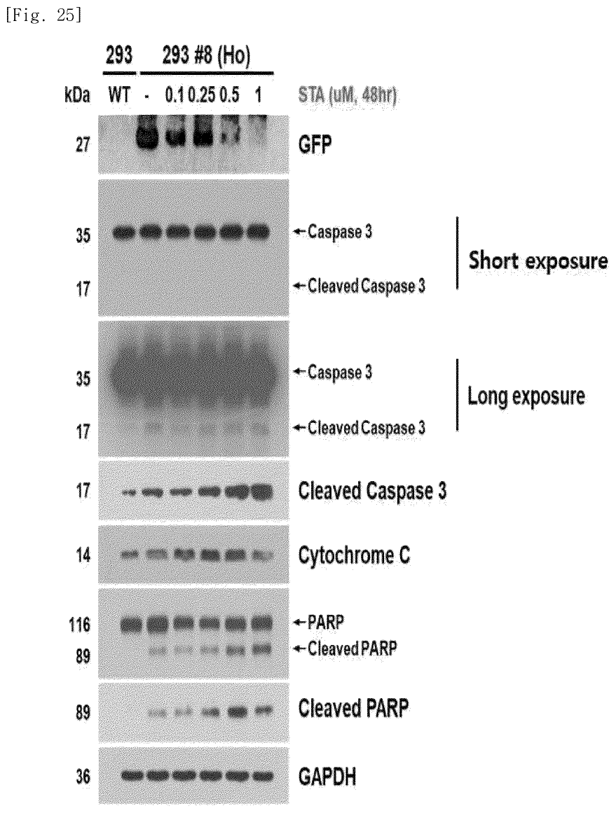

In addition, as shown in FIG. 25, it was confirmed that the expression of cell damage-related proteins such as Cleaved Caspase 3, Cytochrome C, and Cleaved PARP increased as the treatment concentration of staurosporine increased.

Through this, it was confirmed that the change in expression of green fluorescent protein in #8 cells of the AAVS1-EGFP #4 homo genotype was due to the expression of a cell damage-related protein.

### Example 11. Confirmation of Fluorescence Expression of Transformed Cell Lines and Degree of Cell Damage

#8 Cells of AAVS1-EGFP #4 homo genotype described in Example 7 were treated with staurosporine, a substance that induces apoptosis, and then stained using TUNEL (terminal deoxynucleotidyl transferase dUTP nick end labeling) and DAPI staining, which are fluorescent dyes. DNA damage, one of the characteristics of apoptosis, was observed, and through this, the fluorescence expression of the transformed cell line and the degree of cell damage were confirmed.

Specifically, apoptosis was confirmed in #8 cells of AAVS1-EGFP #4 homo genotype treated with staurosporine using an in situ cell death detection kit, POD (Roche, Germany) using the TUNEL method.

First, #8 cells of AAVS1-EGFP #4 homo genotype were seeded in a medium, cultured for 24 hours, treated with 0.5 μM and 1 μM staurosporine for 48 hours, and cultured for 48 hours.

Thereafter, 4% paraformaldehyde was added thereto, and the cells were fixed at room temperature for 30 minutes, washed three times with PBS, and treated with a permeabilization solution including 0.1% Triton X-100 for 10 minutes. After washing again with PBS three times, terminal deoxynucleotidyl transferase (TDT) and a nucleotide mixture were added and the cells were incubated for 1 hour at 37° C. by blocking light.

Thereafter, the cells were washed twice with PBS, and apoptosis was confirmed by observing the cultured cells under a fluorescence microscope. In particular, a 4,6-diamino-2-phenylindole (DAPI, Sigma) solution was used for counterstaining of cells in which apoptosis did not occur.

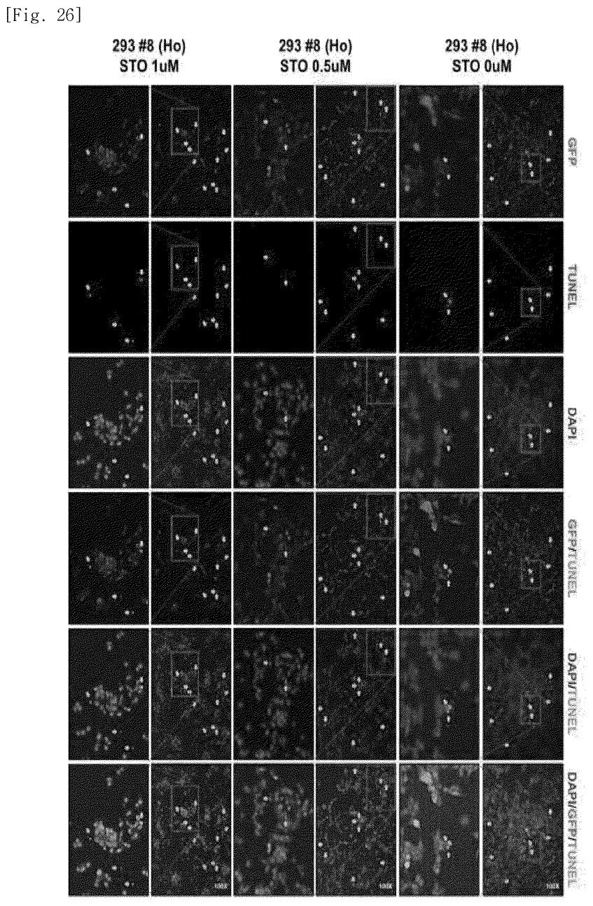

As a result, as shown in FIG. 26, it was confirmed that DNA damage increased in cells in which green fluorescence was decreased by staurosporine treatment, thereby increasing TUNEL fluorescence.

### Example 12. Confirmation of Target Sequence #4 of Blue Fluorescent Protein, Yellow Fluorescent Protein, and Cyan Fluorescent Protein

In order to confirm whether the target sequence #4 is present at the same position in the green fluorescent protein-derived blue fluorescent protein, yellow fluorescent protein, and cyan fluorescent protein, the nucleotide sequences of each fluorescent protein were analyzed. It is known that all fluorescent proteins derived from green fluorescent protein have similar structures, and fluorescence is expressed in different colors because some of the nucleotide sequences encoding green fluorescent protein are different.

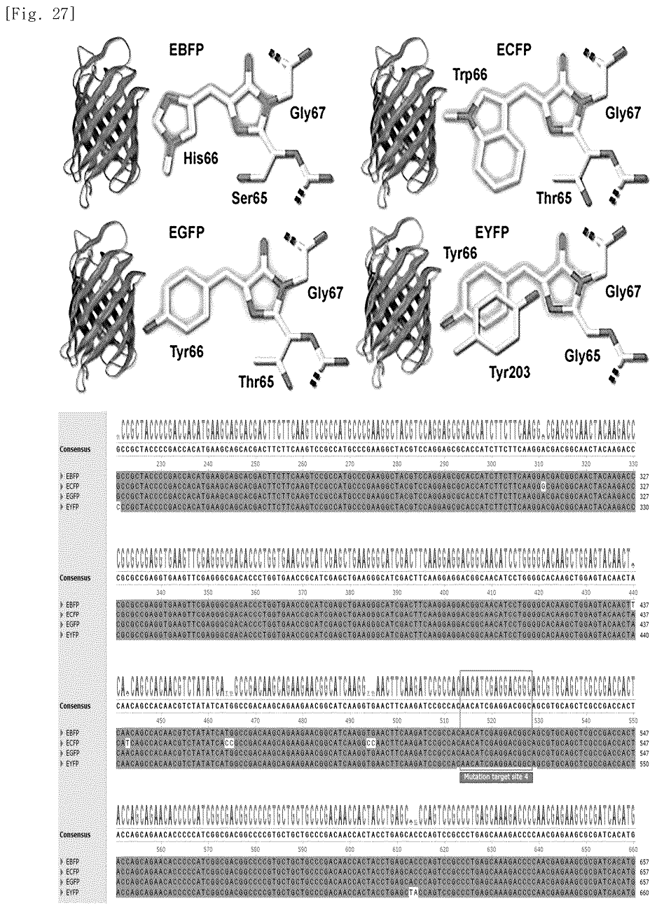

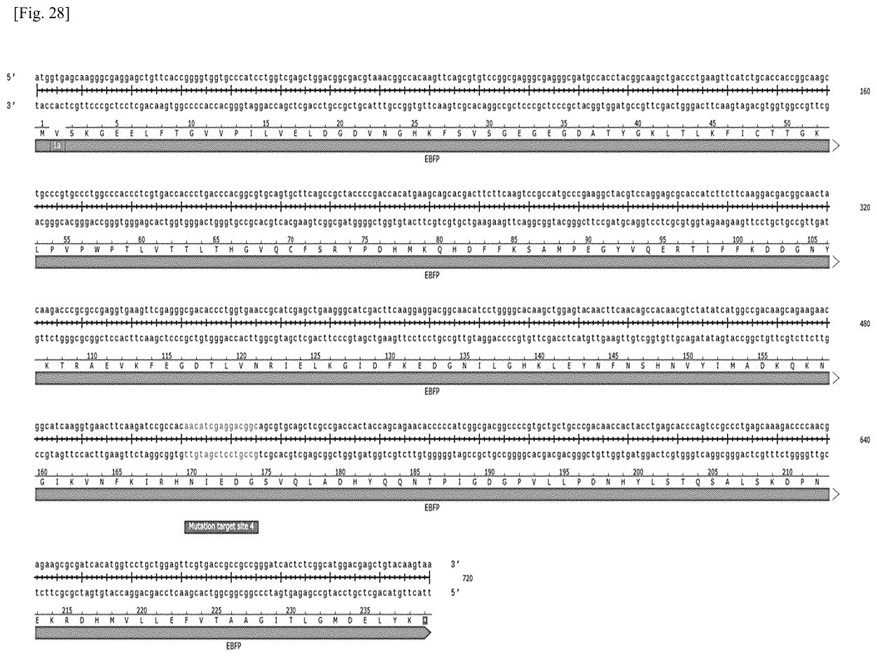

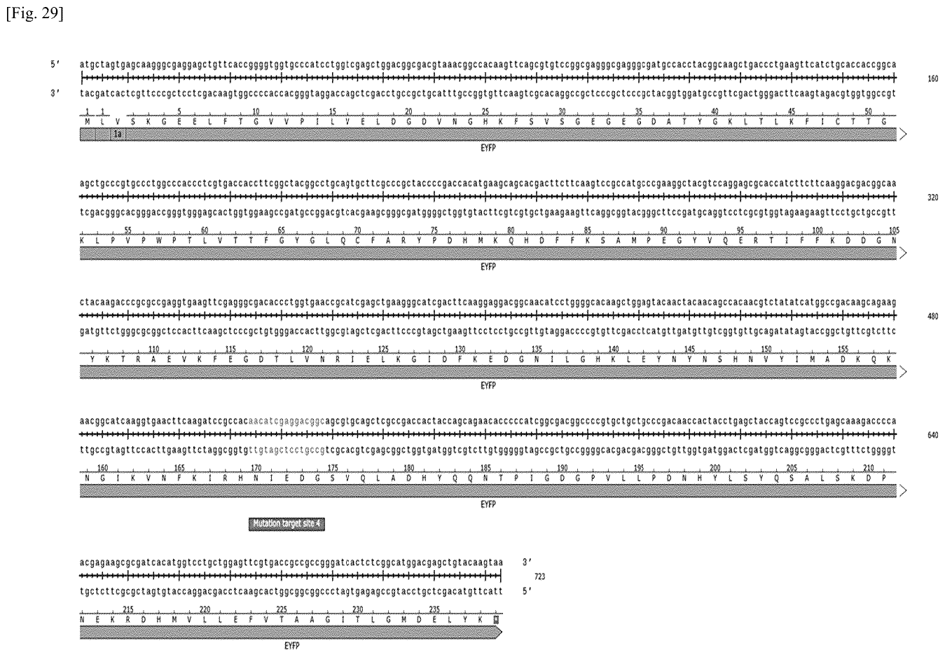

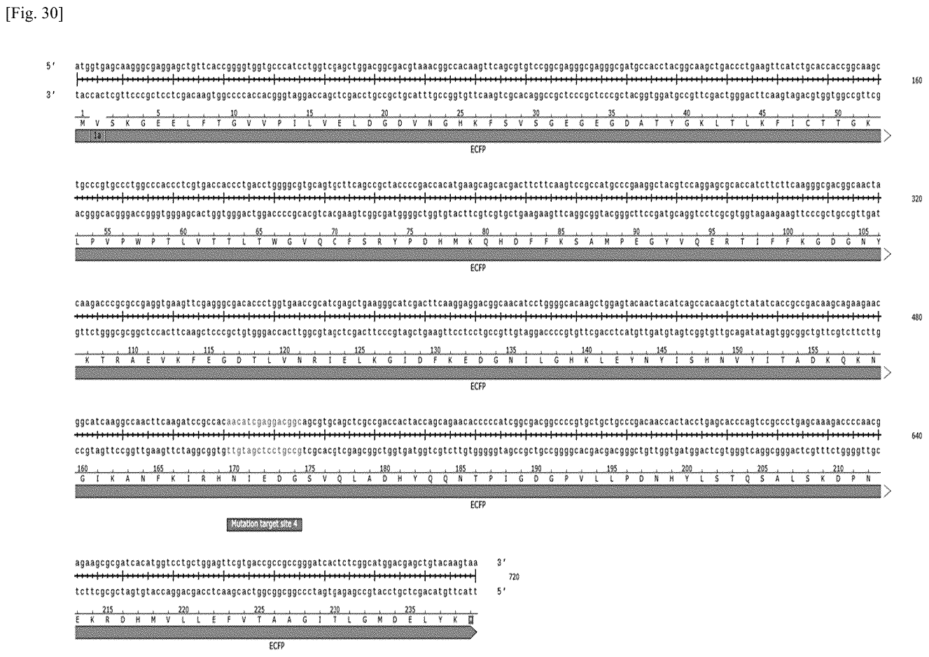

As a result, it was confirmed that even in enhanced blue fluorescent protein (EBFP), enhanced yellow fluorescent protein (EYFP), and enhanced cyan fluorescent protein (ECFP), target sequence #4 was present at the same position as the enhanced green fluorescent protein (FIG. 27). The positions of target sequence #4 in each nucleotide sequence encoding EBFP, EYFP, and ECFP are shown in FIGS. 28 to 30.

Through this, it was found that since the position of target sequence #4 is the same in EBFP, EYFP, and ECFP as in EGFP, mutating target sequence #4 to a nucleotide sequence encoding DEVDG would not affect the function of the fluorescent protein.

### Example 13. Construction of pCAGGS Vector Including Gene Encoding Blue Fluorescent Protein Variant

In the same manner as in Example 2, target sequence #4 of wild-type enhanced blue fluorescent protein (EBFP) was mutated into the GACGAGGTGGACGGC nucleotide sequence (SEQ ID NO: 12) so that the amino acid sequence encoded by these nucleotide sequences could be DEVDG (SEQ ID NO: 13).

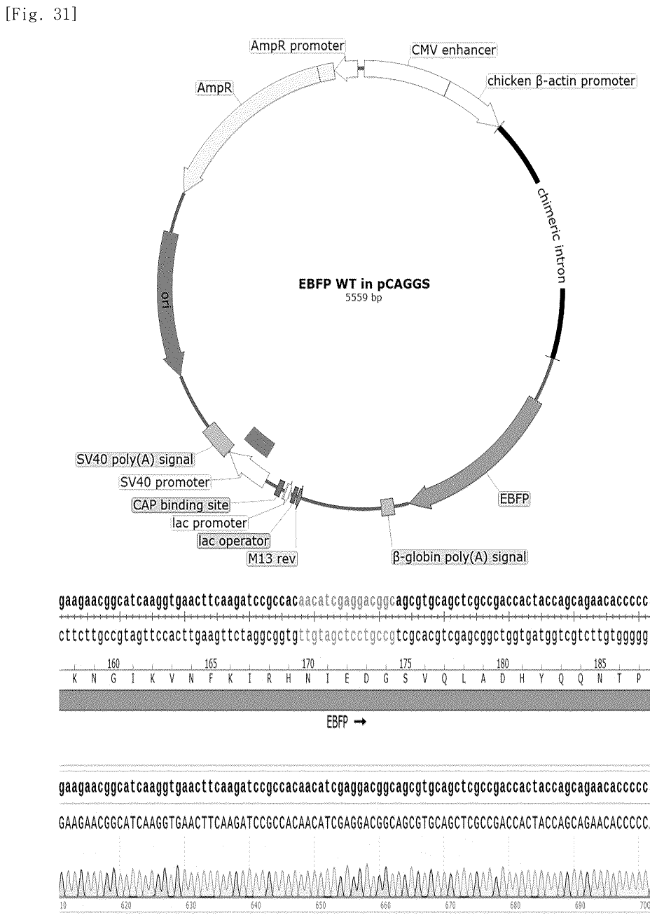

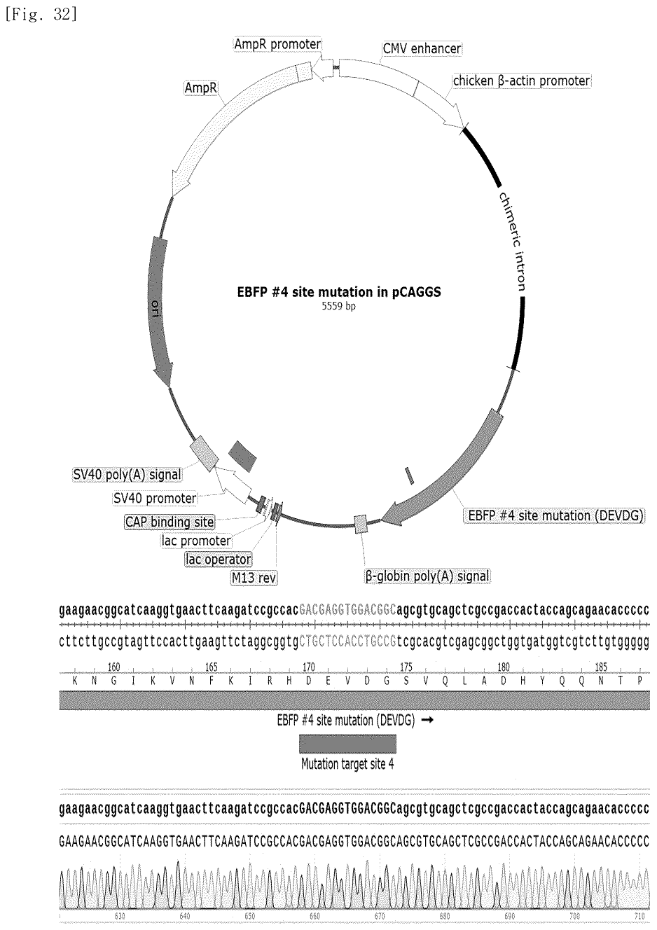

Then, in the same manner as in Example 3, the wild-type EBFP and the EBFP variant genes mutated at position #4 were each cloned into the pCAGGS vector. Since the nucleotide sequences before and after the target sequence #4 of EGFP and EBFP matched, the same in fusion PCR conditions and primers as in the EGFP experiment of Example 3 were used. In particular, individually prepared pCAGGS vectors are shown in FIGS. 31 and 32.

In an LB broth plate, HIT Competent Cells-DH5alpha High 108 (#RH618, RBC) was transformed with the pCAGGS vector into which the genes of wild-type EBFP and variant were inserted, respectively.

### Example 14. Confirmation of Fluorescence Expression of Blue Fluorescent Protein Variants

The expression of the blue fluorescent protein variant of the pCAGGS vector prepared in Example 13 was confirmed.

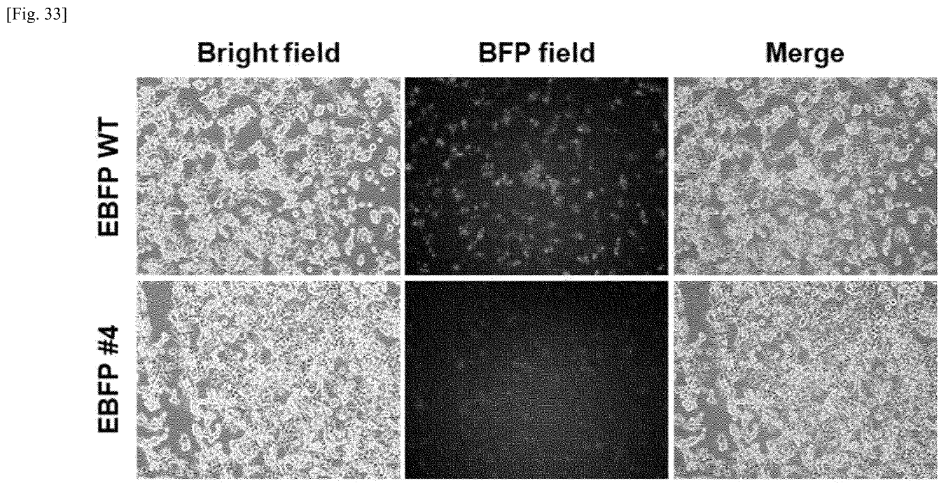

As a result of confirming the expression of the blue fluorescent protein variant in the cell line into which the pCAGGS vector was introduced through a fluorescence microscope, it was confirmed that the blue fluorescent protein variant was normally expressed in the cell line into which pCAGGS including the EBFP #4 mutant gene was introduced (FIG. 33).

Through this, it was confirmed that even in other fluorescent proteins with a consensus nucleotide sequence matching to that of the target sequence #4 of EGFP, when the target sequence #4 was mutated into a gene encoding DEVDG, they could express the same fluorescence as the green fluorescent protein variant, and thus, they could be used to confirm the activity of caspase 3 and caspase 7.

Therefore, not only green fluorescent protein variants but also variants of fluorescent proteins of other colors can be used to observe changes in fluorescence caused by apoptosis in real time.

### Example 15. Confirmation of Monitoring Real-Time Cell Damage-Live Imaging

AAVS1-EGFP #4-8 (homo) genotype 293 cell lines were cultured in Dulbecco's Modified Eagle's Medium (DMEM, #11995, ThermoFisher) supplemented with 10% fetal bovine serum (FBS, #16000, ThermoFisher) and 1% penicillin/streptomycin (#15140, ThermoFisher) in a 5% CO₂, 37° C. incubator. When the cells reached 60-70% confluence in a 100 mm dish, 5×10⁴ cells/well were subcultured onto Lab-Tek™ II chamber Slide™ (#154526PK, Nunc). After 2 days, the cells were treated with staurosporine (STA) at a concentration of 1 μM, and fluorescence was observed at 30-minute intervals for 48 hours using a fluorescence microscope (Leica) equipped with a live cell imaging system.

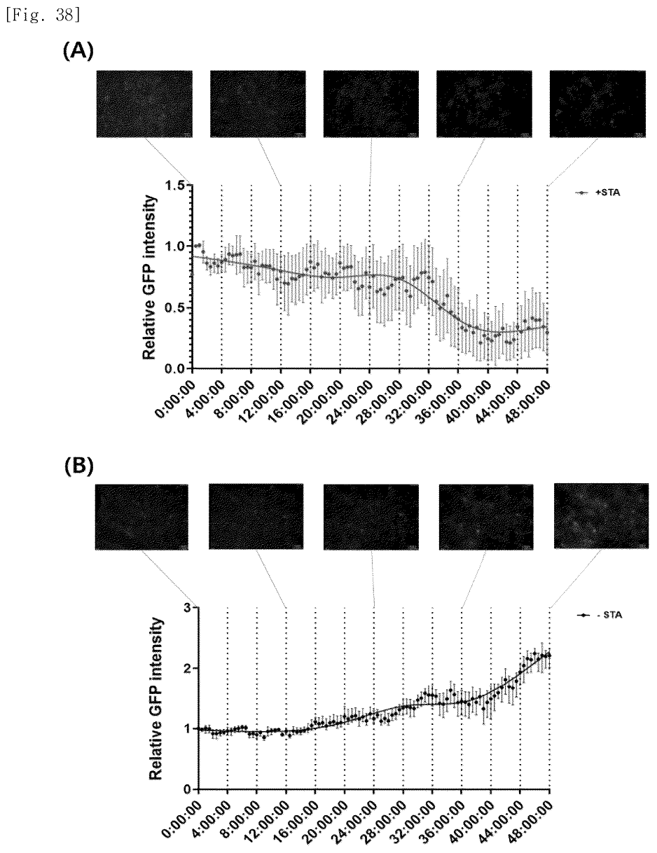

After inducing apoptosis in 293 AAVS1-EGFP #4-8 cells (STA treatment), fluorescence was observed at 30-minute intervals for 48 hours, and a decrease in fluorescence over time was observed (FIG. 38A). In contrast, in the control group (staurosporine-untreated; −STA), fluorescence increased over time, and this increase in fluorescence is believed to be due to cell proliferation (FIG. 38B). Therefore, it was confirmed that the reporter system of the present invention is also suitable for real-time monitoring.

### Example 16. Construction of Transformed Colorectal Cancer Cell Line Introduced with AAVS1-EGFP #4 Vector

The AAVS1 SA-EGFP #4 vector prepared in Example 5 was introduced into colorectal cancer cell lines SW480, SW620, and HCT 116 purchased from ATCC, respectively, to establish transformed cell lines as in Example 6. The cell lines were cultured in a 5% CO₂, 37° C. incubator using Dulbecco's Modified Eagle's Medium (DMEM, #11995, ThermoFisher) supplemented with 10% fetal bovine serum (FBS, #16000, ThermoFisher) and 1% penicillin/streptomycin (#15140, ThermoFisher).

First, SW480, SW620, and HCT 116 cells were cultured in 100 mm dishes. When the cells were 70% to 80% confluence, 5 μg of AAVS1-EGFP #4 vector was introduced into the cells using Lipofectamine 3000 according to the manufacturer's manual. Then, when the cells grew to about 90% confluence, 3 μL of puromycin (#A11138-03, Gibco) was added to 10 mL of culture medium to perform puromycin selection. After 14 days, when the cells grew again to 70% to 80% confluence, cells expressing a green fluorescent protein were selected for the second time using fluorescence-activated cell sorting (FACS). The selected single cells were then seeded into 96-well plates, cultured, and subcultured in 24-well plates to establish transformed cell lines.

In order to confirm whether the AAVS1-EGFP #4 vector was correctly inserted into the colorectal cancer cell lines SW480, SW620, and HCT 116, the genotyping test used in Example 7 was performed.

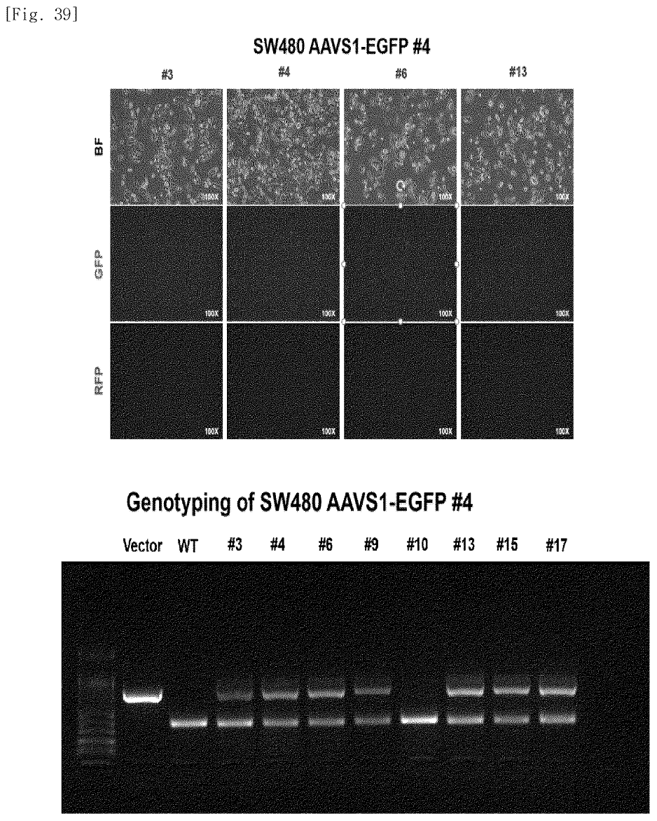

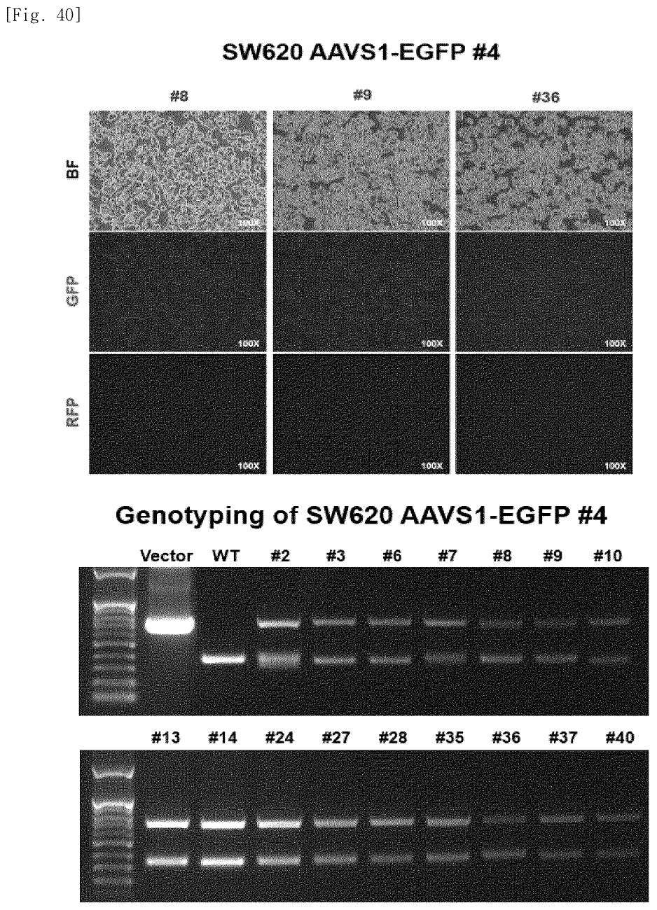

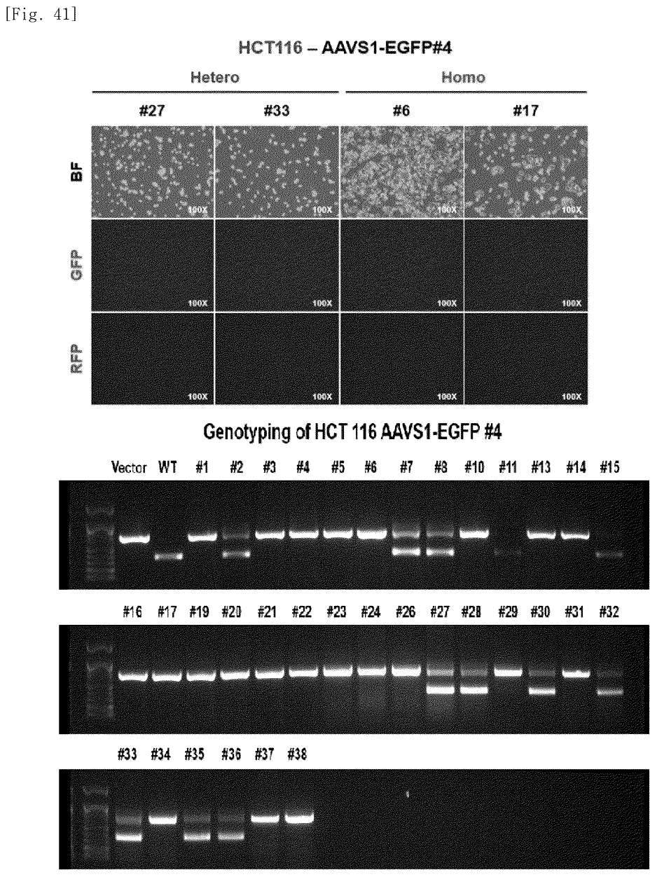

As a result, it was confirmed that cell lines stably expressing a fluorescent protein variant was constructed using the AAVS1-EGFP #4 vector system used in the present invention in each of the colorectal cancer cell lines SW480, SW620, and HCT 116, and hetero and homo cell lines were confirmed through genotyping analysis (FIGS. 39 to 41).

### Example 17. Verification of Cell Damage Reporter Ability of Transformed Colorectal Cancer Cell Lines Introduced with AAVS1-EGFP #4 Vector

In order to verify the cell damage reporter ability, the AAVS1-EGFP #4 expressing colorectal cancer cell lines SW480 and SW620 constructed in Example 16 were treated with staurosporine (STA) at a concentration of 1 μM, and the changes in fluorescence were checked after 24 and 48 hours to confirm whether the reporter was functioning.

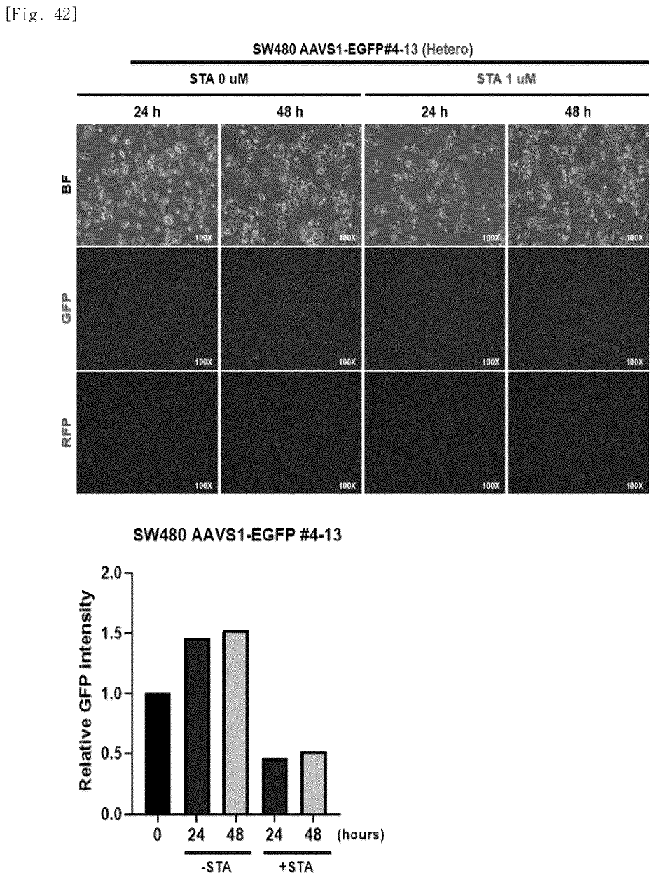

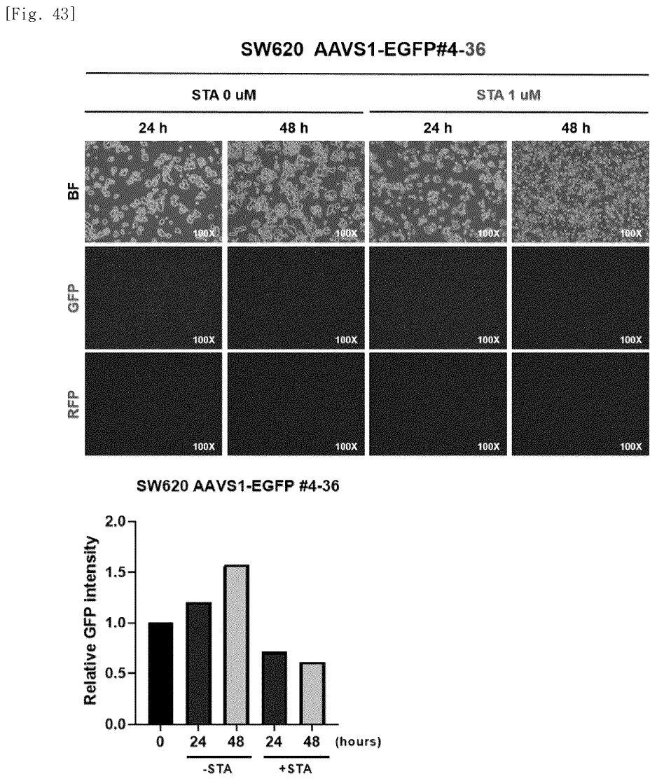

As a result, it was confirmed that when the cells were treated with staurosporine (STA), fluorescence decreased when cell damage occurred compared to the control group (staurosporine untreated; −STA) (FIGS. 42 and 43).

### Comparative Example 1. Confirmation of Non-Uniform Fluorescence Expression in Transformed Cell Line Introduced with pCAGGS-EGFP #4 Vector

The EGFP #4 mutant gene was cloned into the pCAGGS vector to produce the pCAGGS-EGFP #4 vector, which was then introduced into HEK293 cells derived from human embryonic kidney, and the level of fluorescence expression was confirmed.

First, HEK293 cells were seeded into a 100 mm culture dish, and when the cells were cultured to about 70% to 80% confluence, 5 μg of pCAGGS-EGFP #4 vector was introduced into the cells using a 4D-Nucleofector™ device according to the SF kit, CM-130 program, and the manufacturer's manual.

Subsequently, the expression of a green fluorescent protein variant was confirmed in cell lines introduced with each pCAGGS-EGFP #4 vector using fluorescence microscopy and flow cytometry.

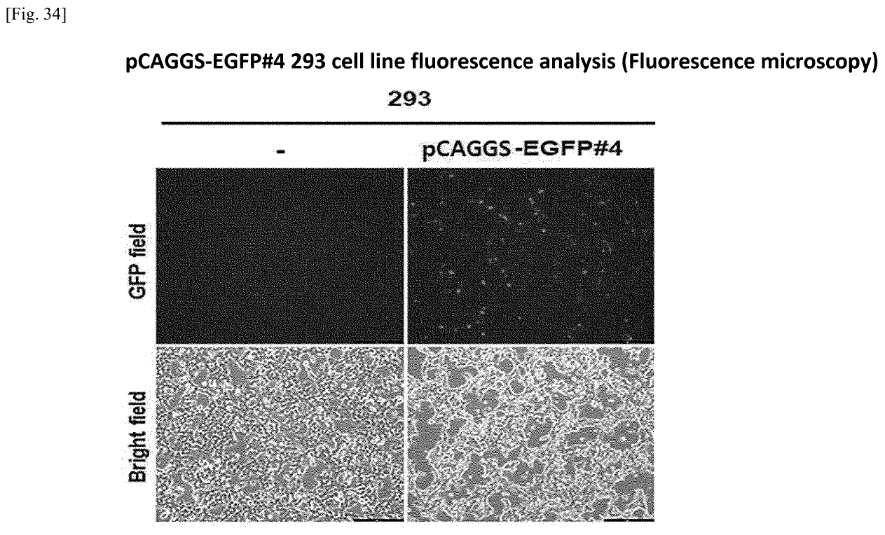

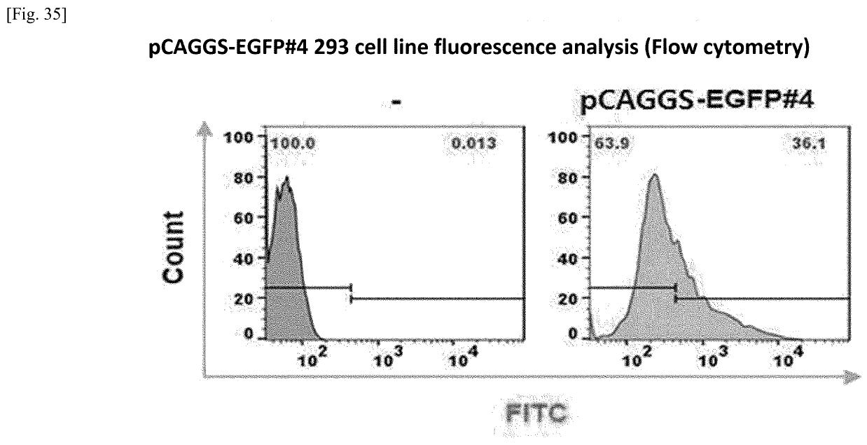

As a result, as shown in FIGS. 34 and 35, it was confirmed that a green fluorescent protein variant was expressed normally but non-uniformly in the cell lines introduced with the pCAGGS-EGFP #4 vector.

Through this, it was confirmed that there are many cells that quantitatively express fluorescence non-uniformly in the cell lines introduced with the pCAGGS-EGFP #4 vector, and that it is difficult to uniformly express a green fluorescent protein variant when the pCAGGS vector is used.

### Comparative Example 2. Confirmation of Level of Fluorescence Expression in Transformed Cell Lines Introduced with pCAGFP Vector

A green fluorescent protein variant was prepared by inserting a caspase recognition sequence (DEVD) between a green fluorescent protein and a quenching peptide (QP) sequence, and the mutant gene was cloned into a Caspase Activatable-GFP (CA-GFP) (pCAGFP #32748, Addgene) vector to prepare a vector system in which fluorescence is expressed when a green fluorescent protein variant is cleaved by caspase 3 or caspase 7 (J Biol Chem. 2011 Jul. 15; 286 (28): 24977-86).

After introducing the prepared vector into HEK293 cells derived from human embryonic kidney, the changes in fluorescence expression were confirmed by treating the cells with staurosporine at a concentration of 1 μM.

Specifically, after HEK293 cells were seeded onto a 100 mm culture dish, when the cells were cultured to about 70% to 80% confluence, 5 μg of pCAGFP vector was introduced into the cells using a 4D-Nucleofector™ device according to the SF kit, CM-130 program, and the manufacturer's manual.

After staurosporine treatment on the cell lines introduced with the pCAGFP vector, the expression of a green fluorescent protein variant was confirmed through fluorescence microscopy and flow cytometry.

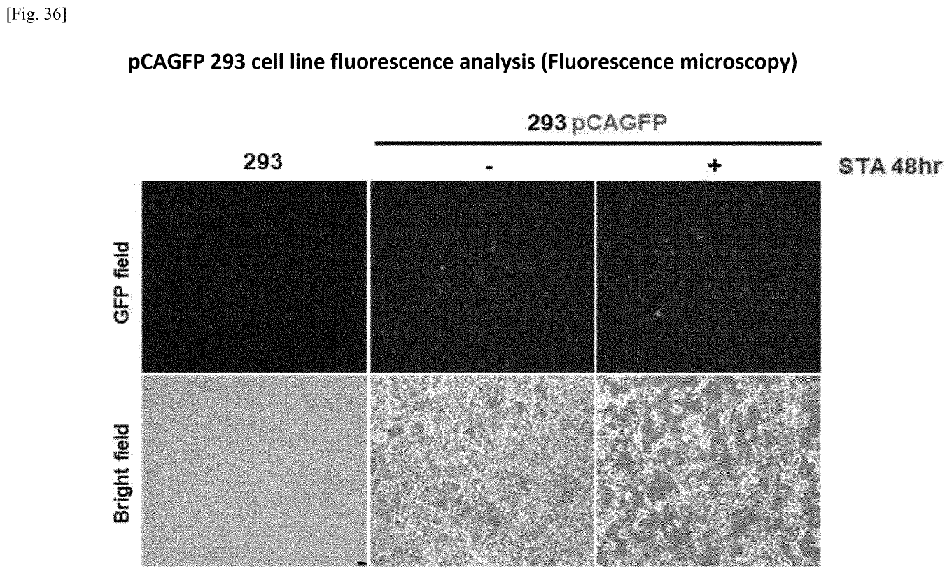

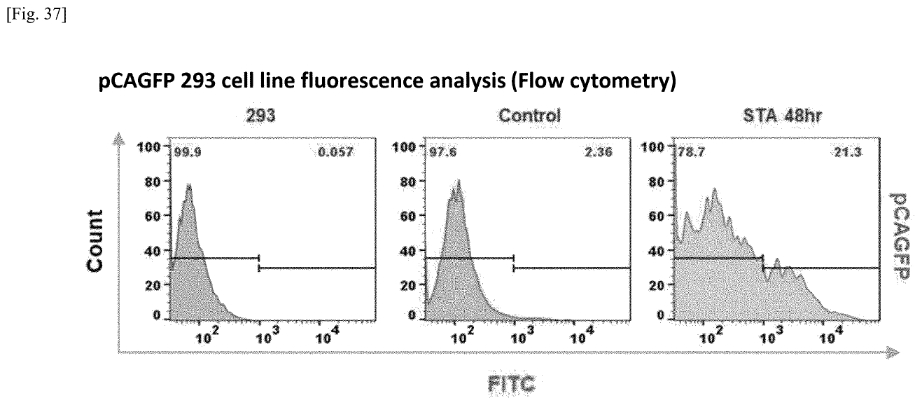

As a result, as shown in FIGS. 36 and 37, it was confirmed that green fluorescence expression increased after treatment with 1 μM staurosporine in a cell line into which the pCAGFP vector was introduced, but fluorescence was not uniformly observed in all cells, and it was confirmed that fluorescence was already strongly expressed before treatment with staurosporine in some cell lines.

Through this, it was confirmed that it is difficult to accurately determine the presence or absence of cell damage caused by fluorescence expression in the above vector system.

1: A fluorescent protein (FP) variant in which fluorescence expression changes in damaged or dead cells, wherein the fluorescent protein variant comprises, in the hinge region that does not affect the function of the fluorescent protein, an amino acid sequence that is cleaved by a caspase.

2: The fluorescent protein variant of claim 1, wherein the fluorescent protein is a green fluorescent protein (GFP), a blue fluorescent protein (BFP), a yellow fluorescent protein (YFP), or a cyan fluorescent protein (CFP).

3: The fluorescent protein variant of claim 1, wherein the amino acid sequence that is cleaved by a caspase is aspartic acid-glutamic acid-valine-aspartic acid-glycine (DEVDG) or aspartic acid-glutamic acid-valine-aspartic acid (DEVD).

4: The fluorescent protein variant of claim 1, wherein an asparagine-isoleucine-glutamic acid (NIE) sequence in the amino acid sequence of a fluorescent protein before mutation is substituted with an aspartic acid-glutamic acid-valine (DEV) sequence.

5: The fluorescent protein variant of claim 4, wherein the asparagine-isoleucine-glutamic acid (NIE) sequence is the 171^{st} to 173^{rd} amino acid of the amino acid sequence represented by SEQ ID NO: 1, the 171ₛₜ to 173^{rd} amino acid of the amino acid sequence represented by SEQ ID NO: 33, the 172^{nd} to 174^{th} amino acid of the amino acid sequence represented by SEQ ID NO: 34, or the 171^{st} to 173^{rd} amino acid of the amino acid sequence represented by SEQ ID NO: 35.

6: The fluorescent protein variant of claim 1, wherein the caspase is caspase 3 or caspase 7.

7: A polynucleotide encoding the fluorescent protein variant of claim 1.

8: A recombinant vector comprising the polynucleotide of claim 7.

9: The recombinant vector of claim 8, wherein the vector is an adeno-associated virus integration site 1 (AAVS1) vector.

10: The recombinant vector of claim 8, wherein the polynucleotide is a nucleotide sequence represented by SEQ ID NO: 14, a nucleotide sequence represented by SEQ ID NO: 39, a nucleotide sequence represented by SEQ ID NO: 40, or a nucleotide sequence represented by SEQ ID NO: 41.

11: A composition for detecting cell damage or apoptosis, comprising the fluorescent protein variant of claim 1, a polynucleotide encoding the fluorescent protein variant, or a recombinant vector comprising the polynucleotide as an active ingredient.

12: A transformed cell transformed with the recombinant vector of claim 8.

13: A method for evaluating drug toxicity, comprising:
i) treating the transformed cell of claim 12 with a sample;
ii) measuring fluorescence expression of the cell treated with the sample; and
iii) determining that the sample has cytotoxicity against the cell when the fluorescence expression changes.

14: The method of claim 13, wherein the change in fluorescence expression in step iii) above is due to the activity of caspase 3 or caspase 7.
